(19) United States
(12) Patent Application Publication
(10) Pub. No.: US 2020/0024619 A1
(43) Pub. Date: Jan. 23, 2020

(54) IMPROVED GLYCEROL FREE ETHANOL PRODUCTION

(71) Applicant: DSM IP Assets B.V., Heerlen (NL)

(72) Inventors: Paulus Petrus DE WAAL, Echt (NL); Jozef Petrus Johannes SCHMITZ, Echt (NL); Hans Marinus Charles Johannes DE BRUIJN, Echt (NL)

(21) Appl. No.: 16/495,638

(22) PCT Filed: Mar. 20, 2018
(86) PCT No.: PCT/EP2018/056969

§ 371 Date: Sep. 19, 2019

(30) Foreign Application Priority Data
Mar. 21, 2017 (EP) 17162006.5
Sep. 26, 2017 (EP) 17193047.2

(51) Int. Cl. C12P 7/06; C12N 9/04; C12N 9/02; C12N 9/12; C12N 9/88

(52) U.S. Cl. CPC C12P 7/06; C12N 9/0006; C12N 9/0008; C12N 9/1205; C12Y 401/02009; C12Y 101/01006; C12Y 102/0101; C12Y 207/01029; C12N 9/88; C12Y 101/01002

(57) ABSTRACT

The invention relates to a recombinant cell, preferably a yeast cell comprising one or more genes coding for an enzyme having glycerol dehydrogenase activity, one or more genes coding dihydroxyacetone kinase (E.C. 2.7.1.28 and/or E.C. 2.7.1.29); one or more genes coding for an enzyme in an acetyl-CoA-production pathway and one or more genes coding for an enzyme having at least NAD⁺ dependent acetylating acetaldehyde dehydrogenase activity (EC 1.2.1.10 or EC 1.1.1.2), and optionally one or more genes coding for a glycerol transporter. This cell can be used for the production of ethanol and advantageously produces little or no glycerol.

### FIELD

The invention relates to a recombinant cell suitable for ethanol production, the use of this cell for the preparation of ethanol and/or succinic acid, and a process for preparing fermentation product using said recombinant cell.

### BACKGROUND

Microbial fermentation processes are applied for industrial production of a broad and rapidly expanding range of chemical compounds from renewable carbohydrate feedstocks. Especially in anaerobic fermentation processes, redox balancing of the cofactor couple NADH/NAD⁺ can cause important constraints on product yields. This challenge is exemplified by the formation of glycerol as major by-product in the industrial production of—for instance—fuel ethanol by Saccharomyces cerevisiae, a direct consequence of the need to reoxidize NADH formed in biosynthetic reactions. Ethanol production by Saccharomyces cerevisiae is currently, by volume, the single largest fermentation process in industrial biotechnology, but various other compounds, including other alcohols, carboxylic acids, isoprenoids, amino acids etc., are currently produced in industrial biotechnological processes. For conventional fermentative production of fuel ethanol, such as from corn starch and cane sugar, sugars predominantly occur as dimers or polymers of hexose sugars, which upon release in monosaccharides after pretreatment and enzymatic hydrolysis by different forms of glucohydrolases can be efficiently and rapidly fermented by Saccharomyces cerevisiae. Cellulosic or second generation bioethanol is produced from e.g. lignocellulosic fractions of plant biomass that is hydrolyzed intro free monomeric sugars, such as hexoses and pentoses, for fermentation into ethanol. Apart from the sugar release during pretreatment and hydrolysis of the biomass, some toxic by-products are formed depending on several pretreatment parameters, such as temperature, pressure and pre-treatment time. Various approaches have been proposed to improve the fermentative properties of organisms used in industrial biotechnology by genetic modification. A major challenge relating to the stoichiometry of yeast-based production of ethanol, but also of other compounds, is that substantial amounts of NADH-dependent side-products (in particular glycerol) are generally formed as a by-product, especially under anaerobic and oxygen-limited conditions or under conditions where respiration is otherwise constrained or absent. It has been estimated that, in typical industrial ethanol processes, up to about 4 wt % of the sugar feedstock is converted into glycerol (Nissen et al. Yeast 16 (2000) 463-474). Under conditions that are ideal for anaerobic growth, the conversion into glycerol may even be higher, up to about 10%.

Glycerol production under anaerobic conditions is primarily linked to redox metabolism. During anaerobic growth of S. cerevisiae, sugar dissimilation occurs via alcoholic fermentation.

In this process, the NADH formed in the glycolytic glyceraldehyde-3-phosphate dehydrogenase reaction is re-oxidized by converting acetaldehyde, formed by decarboxylation of pyruvate to ethanol via NAD³⁰ dependent alcohol dehydrogenase. The fixed stoichiometry of this redox-neutral dissimilatory pathway causes problems when a net reduction of NAD⁺ to NADH occurs elsewhere in metabolism (e.g. biomass formation). Under anaerobic conditions, NADH re-oxidation in S. cerevisiae is strictly dependent on reduction of sugar to glycerol. Glycerol formation is initiated by reduction of the glycolytic intermediate dihydroxyacetone phosphate (DHAP) to glycerol 3-phosphate (glycerol-3P), a reaction catalyzed by NAD⁺ dependent glycerol 3-phosphate dehydrogenase. Subsequently, the glycerol 3-phosphate formed in this reaction is hydrolysed by glycerol-3-phosphatase to yield glycerol and inorganic phosphate. Consequently, glycerol is a major by-product during anaerobic production of ethanol by S. cerevisiae, which is undesired as it reduces overall conversion of sugar to ethanol. Further, the presence of glycerol in effluents of ethanol production plants may impose costs for waste-water treatment.

In the literature, however, several different approaches have been reported that could help to reduce the byproduct formation of glycerol and divert carbon to ethanol resulting in a ethanol yield increase per gram of fermented carbohydrate.

Sonderegger et al (2004, Applied and Environmental Microbiology, 70(5), pp. 2892-2897) disclosed the heterologous expression of phosphotransacetylase and acetaldehyde dehydrogenase in a xylose-fermenting S. cerevisiae strain.

WO2014/081803 describes a recombinant microorganism expressing a heterologous phosphoketolase, phosphotransacetylase or acetate kinase and bifunctional acetaldeyde-alcohol dehydrogenase. Additionally, the recombinants described in the examples lacked glycerol-3-phosphate dehydrogenase activity (gpd1/gpd2 double deletion strain) or formate dehydrogenase activity (fdh1/fdh2 double deletion strain).

WO2015/148272 described a recombinant S. cerevisiae strain expressing a heterologous phosphoketolase, phosphotransacetylase and acetylating acetaldehyde dehydrogenase achieving an ethanol yield increase. Inventors also displayed with reducing the glycerol biosynthetic pathway (shown in embodiment with deletion of gpd1) that higher yields can be achieved. However, inventors mentioned that glucose fermentation rates were slower strains with reduced glycerol synthesis pathway.

### SUMMARY OF THE INVENTION

The invention provides a recombinant cell, preferably a yeast cell comprising:
one or more genes coding for an enzyme having glycerol dehydrogenase activity;
one or more genes coding dihydroxyacetone kinase (E.C. 2.7.1.28 and/or E.C. 2.7.1.29);
one or more genes coding for an enzyme in an acetyl-CoA-production pathway; and
one or more genes coding for an enzyme having at least NAD⁺ dependent acetylating acetaldehyde dehydrogenase activity (EC 1.2.1.10 or EC 1.1.1.2); and optionally
one or more genes coding for a glycerol transporter.

This recombinant cell can be advantageously used to produce ethanol from cellulosic or starch-based material with high ethanol yield and little or even no glycerol production. Glycerol may still be produced, but is—at least partially—converted to ethanol. Another advantage of this cell is that is has a good growth rate, e.g. when grown under industrial conditions such as on corn mash.

### DETAILED DESCRIPTION

The term “a” or “an” as used herein is defined as “at least one” unless specified otherwise. When referring to a noun (e.g. a compound, an additive, etc.) in the singular, the plural is meant to be included. Thus, when referring to a specific moiety, e.g. “gene”, this means “at least one” of that gene, e.g. “at least one gene”, unless specified otherwise. The term ‘or’ as used herein is to be understood as ‘and/or’.

When referring to a compound of which several isomers exist (e.g. a D and an L enantiomer), the compound in principle includes all enantiomers, diastereomers and cis/trans isomers of that compound that may be used in the particular method of the invention; in particular when referring to such as compound, it includes the natural isomer(s).

The term ‘fermentation’, ‘fermentative’ and the like is used herein in a classical sense, i.e. to indicate that a process is or has been carried out under anaerobic conditions. Anaerobic conditions are herein defined as conditions without any oxygen or in which essentially no oxygen is consumed by the cell, in particular a yeast cell, and usually corresponds to an oxygen consumption of less than 5 mmol/l/h, in particular to an oxygen consumption of less than 2.5 mmol/l.h⁻¹, or less than 1 mmol/l/h. More preferably 0 mmol/L/h is consumed (i.e. oxygen consumption is not detectable. This usually corresponds to a dissolved oxygen concentration in the culture broth of less than 5% of air saturation, in particular to a dissolved oxygen concentration of less than 1% of air saturation, or less than 0.2% of air saturation.

The term “cell” refers to a eukaryotic or prokaryotic organism, preferably occuring as a single cell. The cell may be selected from the group of fungi, yeasts, euglenoids, archaea and bacteria.

The cell may in particular be selected from the group of genera consisting of yeast.

The term “yeast” or “yeast cell” refers to a phylogenetically diverse group of single-celled fungi, most of which are in the division of Ascomycota and Basidiomycota. The budding yeasts (“true yeasts”) are classified in the order Saccharomycetales, with Saccharomyces cerevisiae as the most well-known species.

The term “recombinant (cell)” or “recombinant micro-organism” as used herein, refers to a strain (cell) containing nucleic acid which is the result of one or more genetic modifications using recombinant DNA technique(s) and/or another mutagenic technique(s). In particular a recombinant cell may comprise nucleic acid not present in a corresponding wild-type cell, which nucleic acid has been introduced into that strain (cell) using recombinant DNA techniques (a transgenic cell), or which nucleic acid not present in said wild-type is the result of one or more mutations—for example using recombinant DNA techniques or another mutagenesis technique such as UV-irradiation—in a nucleic acid sequence present in said wild-type (such as a gene encoding a wild-type polypeptide) or wherein the nucleic acid sequence of a gene has been modified to target the polypeptide product (encoding it) towards another cellular compartment. Further, the term “recombinant (cell)” in particular relates to a strain (cell) from which DNA sequences have been removed using recombinant DNA techniques.

The term “transgenic (yeast) cell” as used herein, refers to a strain (cell) containing nucleic acid not naturally occurring in that strain (cell) and which has been introduced into that strain (cell) using recombinant DNA techniques, i.e. a recombinant cell).

The term “mutated” as used herein regarding proteins or polypeptides means that at least one amino acid in the wild-type or naturally occurring protein or polypeptide sequence has been replaced with a different amino acid, inserted or deleted from the sequence via mutagenesis of nucleic acids encoding these amino acids. Mutagenesis is a well-known method in the art, and includes, for example, site-directed mutagenesis by means of PCR or via oligonucleotide-mediated mutagenesis as described in Sambrook et al., Molecular Cloning-A Laboratory Manual, 2nd ed., Vol. 1-3 (1989). The term “mutated” as used herein regarding genes means that at least one nucleotide in the nucleic acid sequence of that gene or a regulatory sequence thereof, has been replaced with a different nucleotide, or has been deleted from the sequence via mutagenesis, resulting in the transcription of a protein sequence with a qualitatively of quantitatively altered function or the knock-out of that gene.

In the context of this invention an “altered gene” has the same meaning as a mutated gene.

The term “gene”, as used herein, refers to a nucleic acid sequence containing a template for a nucleic acid polymerase, in eukaryotes, RNA polymerase II. Genes are transcribed into mRNAs that are then translated into protein.

The term “nucleic acid” as used herein, includes reference to a deoxyribonucleotide or ribonucleotide polymer, i.e. a polynucleotide, in either single or double-stranded form, and unless otherwise limited, encompasses known analogues having the essential nature of natural nucleotides in that they hybridize to single-stranded nucleic acids in a manner similar to naturally occurring nucleotides (e. g., peptide nucleic acids). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulphation, gamma-carboxylation of glutamic acid residues, hydroxylation and ADP-ribosylation.

When an enzyme is mentioned with reference to an enzyme class (EC), the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.qmul.ac.uk/iubmb/enzyme/. Other suitable enzymes that have not (yet) been classified in a specified class but may be classified as such, are meant to be included.

If referred herein to a protein or a nucleic acid sequence, such as a gene, by reference to a accession number, this number in particular is used to refer to a protein or nucleic acid sequence (gene) having a sequence as can be found via www.ncbi.nlm.nih.gov/, (as available on 14 Jun. 2016) unless specified otherwise.

Every nucleic acid sequence herein that encodes a polypeptide also, by reference to the genetic code, describes every possible silent variation of the nucleic acid. The term “conservatively modified variants” applies to both amino acid and nucleic acid sequences. With respect to particular nucleic acid sequences, the term conservatively modified variants refers to those nucleic acids which encode identical or conservatively modified variants of the amino acid sequences due to the degeneracy of the genetic code. The term “degeneracy of the genetic code” refers to the fact that a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation.

The term “functional homologue” (or in short “homologue”) of a polypeptide having a specific sequence (e.g. “SEQ ID NO: X”), as used herein, refers to a polypeptide comprising said specific sequence with the proviso that one or more amino acids are substituted, deleted, added, and/or inserted, and which polypeptide has (qualitatively) the same enzymatic functionality for substrate conversion. This functionality may be tested by use of an assay system comprising a recombinant cell comprising an expression vector for the expression of the homologue in yeast, said expression vector comprising a heterologous nucleic acid sequence operably linked to a promoter functional in the yeast and said heterologous nucleic acid sequence encoding the homologous polypeptide of which enzymatic activity for converting acetyl-Coenzyme A to acetaldehyde in the cell is to be tested, and assessing whether said conversion occurs in said cells. Candidate homologues may be identified by using in silico similarity analyses. A detailed example of such an analysis is described in Example 2 of WO02009/013159. The skilled person will be able to derive there from how suitable candidate homologues may be found and, optionally upon codon(pair) optimization, will be able to test the required functionality of such candidate homologues using a suitable assay system as described above. A suitable homologue represents a polypeptide having an amino acid sequence similar to a specific polypeptide of more than 50%, preferably of 60% or more, in particular of at least 70%, more in particular of at least 80%, at least 90%, at least 95%, at least 97%, at least 98% or at least 99% and having the required enzymatic functionality. With respect to nucleic acid sequences, the term functional homologue is meant to include nucleic acid sequences which differ from another nucleic acid sequence due to the degeneracy of the genetic code and encode the same polypeptide sequence.

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences.

Amino acid or nucleotide sequences are said to be homologous when exhibiting a certain level of similarity. Two sequences being homologous indicate a common evolutionary origin. Whether two homologous sequences are closely related or more distantly related is indicated by “percent identity” or “percent similarity”, which is high or low respectively. Although disputed, to indicate “percent identity” or “percent similarity”, “level of homology” or “percent homology” are frequently used interchangeably. A comparison of sequences and determination of percent identity between two sequences can be accomplished using a mathematical algorithm. The skilled person will be aware of the fact that several different computer programs are available to align two sequences and determine the homology between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent identity between two amino acid sequences can be determined using the Needleman and Wunsch algorithm for the alignment of two sequences. (Needleman, S. B. and Wunsch, C. D. (1970) J. Mol. Biol. 48, 443-453). The algorithm aligns amino acid sequences as well as nucleotide sequences. The Needleman-Wunsch algorithm has been implemented in the computer program

NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp276-277, http://emboss.bioinformatics.nl/). For protein sequences, EBLOSUM62 is used for the substitution matrix. For nucleotide sequences, EDNAFULL is used. Other matrices can be specified. The optional parameters used for alignment of amino acid sequences are a gap-open penalty of 10 and a gap extension penalty of 0.5. The skilled person will appreciate that all these different parameters will yield slightly different results but that the overall percentage identity of two sequences is not significantly altered when using different algorithms.

The homology or identity is the percentage of identical matches between the two full sequences over the total aligned region including any gaps or extensions. The homology or identity between the two aligned sequences is calculated as follows: Number of corresponding positions in the alignment showing an identical amino acid in both sequences divided by the total length of the alignment including the gaps. The identity defined as herein can be obtained from NEEDLE and is labelled in the output of the program as “IDENTITY”.

The homology or identity between the two aligned sequences is calculated as follows: Number of corresponding positions in the alignment showing an identical amino acid in both sequences divided by the total length of the alignment after subtraction of the total number of gaps in the alignment. The identity defined as herein can be obtained from NEEDLE by using the NOBRIEF option and is labeled in the output of the program as “longest-identity”.

A variant of a nucleotide or amino acid sequence disclosed herein may also be defined as a nucleotide or amino acid sequence having one or several substitutions, insertions and/or deletions as compared to the nucleotide or amino acid sequence specifically disclosed herein (e.g. in de the sequence listing).

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. In an embodiment, conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. In an embodiment, conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

Nucleotide sequences of the invention may also be defined by their capability to hybridise with parts of specific nucleotide sequences disc losed herein, respectively, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6 ×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

“Expression” refers to the transcription of a gene into structural RNA (rRNA, tRNA) or messenger RNA (mRNA) with subsequent translation into a protein.

As used herein, “heterologous” in reference to a nucleic acid or protein is a nucleic acid or protein that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

The term “heterologous expression” refers to the expression of heterologous nucleic acids in a host cell. The expression of heterologous proteins in eukaryotic host cell systems such as yeast are well known to those of skill in the art. A polynucleotide comprising a nucleic acid sequence of a gene encoding an enzyme with a specific activity can be expressed in such a eukaryotic system. In some embodiments, transformed/transfected cells may be employed as expression systems for the expression of the enzymes. Expression of heterologous proteins in yeast is well known. Sherman, F., et al., Methods in Yeast Genetics, Cold Spring Harbor Laboratory (1982) is a well-recognized work describing the various methods available to express proteins in yeast. Two widely utilized yeasts are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

As used herein “promoter” is a DNA sequence that directs the transcription of a (structural) gene. Typically, a promoter is located in the 5′-region of a gene, proximal to the transcriptional start site of a (structural) gene. Promoter sequences may be constitutive, inducible or repressible. In an embodiment there is no (external) inducer needed.

The term “vector” as used herein, includes reference to an autosomal expression vector and to an integration vector used for integration into the chromosome.

The term “expression vector” refers to a DNA molecule, linear or circular, that comprises a segment encoding a polypeptide of interest under the control of (i.e. operably linked to) additional nucleic acid segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both. In particular an expression vector comprises a nucleic acid sequence that comprises in the 5′ to 3′ direction and operably linked: (a) a yeast-recognized transcription and translation initiation region, (b) a coding sequence for a polypeptide of interest, and (c) a yeast-recognized transcription and translation termination region. “Plasmid” refers to autonomously replicating extrachromosomal DNA which is not integrated into a microorganism's genome and is usually circular in nature.

An “integration vector” refers to a DNA molecule, linear or circular, that can be incorporated in a microorganism's genome and provides for stable inheritance of a gene encoding a polypeptide of interest. The integration vector generally comprises one or more segments comprising a gene sequence encoding a polypeptide of interest under the control of (i.e. operably linked to) additional nucleic acid segments that provide for its transcription. Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is nonfunctional in that organism. Integration of the segment comprising the gene of interest may be selected if an appropriate marker is included within that segment.

By “host cell” is meant a cell which contains a vector and supports the replication and/or expression of the vector.

“Transformation” and “transforming”, as used herein, refers to the insertion of an exogenous polynucleotide into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome.

By “disruption” is meant (or includes) all nucleic acid modifications such as nucleotide deletions or substitutions, gene knock-outs, (other) which affect the translation or transcription of the corresponding polypeptide and/or which affect the enzymatic (specific) activity, its substrate specificity, and/or or stability. Such modifications may be targeted on the coding sequence or on the promotor of the gene.

As used herein, “reduced enzymatic activity” can be achieved by modifying one or more genes encoding the targeted enzyme such that the enzyme is expressed considerably less than in the wild-type or such that the gene encodes a polypeptide with reduced activity. Such modifications can be carried out using commonly known biotechnological techniques, and may in particular include one or more knock-out mutations or site-directed mutagenesis of promoter regions or coding regions of the structural genes encoding the targeted enzyme.

The invention provides a recombinant cell, preferably a yeast cell comprising:
one or more genes coding for an enzyme having glycerol dehydrogenase activity;
one or more genes coding dihydroxyacetone kinase (E.C. 2.7.1.28 and/or E.C. 2.7.1.29);
one or more genes coding for an enzyme in an acetyl-CoA-production pathway; and
one or more genes coding for an enzyme having at least NAD⁺ dependent acetylating acetaldehyde dehydrogenase activity (EC 1.2.1.10 or EC 1.1.1.2); and optionally
one or more genes coding for a glycerol transporter.

The inventors have found that such recombinant cell can be advantageously used to produce ethanol from cellulosic or starch-based material with high ethanol yield and little or even no glycerol production. Glycerol may still be produced, but is—at least partially—converted to ethanol. Another advantage of this cell is that is has a good growth rate, e.g. when grown under industrial conditions such as on corn mash.

The recombinant cell comprises one or more (heterologous) genes coding for an enzyme having NAD⁺ linked glycerol dehydrogenase. As used herein, a glycerol dehydrogenase catalyzes at least the following reaction (I):

glycerol+NAD<->glycerone+NADH+H⁺ (I)

Thus, the two substrates of this enzyme are glycerol and NAD⁺, whereas its three products are glycerone, NADH, and H⁺. Glycerone and dihydroxyacetone are herein synonyms.

This enzyme belongs to the family of oxidoreductases, specifically those acting on the CH—OH group of donor with NAD+ or NADP+ as acceptor. The systematic name of this enzyme class is glycerol:NAD+ 2-oxidoreductase. Other names in common use include glycerin dehydrogenase, and NAD+-linked glycerol dehydrogenase. This enzyme participates in glycerolipid metabolism. Structural studies have shown that the enzyme is zinc-dependent with the active site lying between the two domains of the protein.

In an embodiment the enzyme having glycerol dehydrogenase activity is preferably a NAD⁺ linked glycerol dehydrogenase (EC 1.1.1.6). Such enzyme may be from bacterial origin or for instance from fungal origin. An example is gldA from E. coli.

Alternatively, the enzyme having glycerol dehydrogenase activity is a NAD⁺ linked glycerol dehydrogenase (EC 1.1.1.72).

When the recombinant cell is used for ethanol production, which typically takes place under anaerobic conditions, NAD⁺ linked glycerol dehydrogenases are preferred.

In an embodiment the cell comprises one or more genes encoding a heterologous glycerol dehydrogenase represented by amino acid sequence SEQ ID NO:15, 16, 17, or 18 or a functional homologue thereof a having sequence identity of at least 50%, preferably at least 60%, 70%, 75%, 80%. 85%, 90% or 95%.

Examples of suitable glycerol dehydrogenases are listed in table 1(a) to 1(d). At the top of each table the gldA that is BLASTED is mentioned.

TABLE 1(a)
BLAST Query - gldA from Escherichia coli (SEQ ID NO: 15)

| Description | Identity (%) | Accession number |
|---|---|---|
| glycerol dehydrogenase, NAD | 100 | NP_418380.4 |
| [Escherichia coli str. K-12 substr. | | |
| MG1655] | | |
| glycerol dehydrogenase | 99 | YP_002331714.1 |
| [Escherichia coli O127:H6 str. | | |
| E2348/69] | | |
| glycerol dehydrogenase | 94 | WP_006686227.1 |
| [Citrobacter youngae] | | |
| glycerol dehydrogenase | 92 | WP_003840533.1 |
| [Citrobacter freundii] | | |

TABLE 1(b)
BLAST Query - gldA from Klebsiella pneumoniae (SEQ ID NO: 16)

| Description | Identity (%) | Accession number |
|---|---|---|
| glycerol dehydrogenase | 100 | YP_002236495.1 |
| [Klebsiella pneumoniae 342] | | |
| glycerol dehydrogenase | 93 | WP_003024745.1 |
| [Citrobacter freundii] | | |
| Glycerol dehydrogenase (EC 1.1.1.6) | 92 | YP_004590977.1 |
| [Enterobacter aerogenes EA1509E] | | |
| glycerol dehydrogenase | 91 | WP_016241524.1 |
| [Escherichia coli] | | |
| glycerol dehydrogenase | 74 | WP_004701845.1 |
| [Yersinia aldovae] | | |
| glycerol dehydrogenase | 61 | WP_017375113.1 |
| [Enterobacteriaceae bacterium | | |
| LSJC7] | | |
| glycerol dehydrogenase | 60 | WP_006686227.1 |
| [Citrobacter youngae] | | |

TABLE 1(c)
BLAST Query - gldA from Enterococcus aerogenes (SEQ ID NO: 17)

| Description | Identity (%) | Accession number |
|---|---|---|
| glycerol dehydrogenase | 100 | YP_004591726.1 |
| [Enterobacter aerogenes | | |
| KCTC 2190] | | |
| Glycerol dehydrogenase (EC 1.1.1.6) | 99 | YP_007390021.1 |
| [Enterobacter aerogenes | | |
| EA1509E] | | |
| glycerol dehydrogenase | 92 | WP_004203683.1 |
| [Klebsiella pneumoniae] | | |
| glycerol dehydrogenase | 88 | WP_001322519.1 |
| [Escherichia coli] | | |
| glycerol dehydrogenase | 87 | YP_003615506.1 |
| [Enterobacter cloacae subsp. cloacae | | |
| ATCC 13047] | | |

TABLE 1(d)
BLAST Query - gldA from Yersinia aldovae (SEQ ID NO: 18)

| Description | Identity (%) | Accession number |
|---|---|---|
| glycerol dehydrogenase | 100 | WP_004701845.1 |
| [Yersinia aldovae] | | |
| glycerol dehydrogenase | 95 | WP_005189747.1 |
| [Yersinia intermedia] | | |
| glycerol dehydrogenase | 81 | YP_008232202.1 |
| [Serratia liquefaciens ATCC 27592] | | |
| glycerol dehydrogenase | 76 | WP_016241524.1 |
| [Escherichia coli] | | |
| hypothetical protein EAE_03845 | 75 | YP_004590977.1 |
| [Enterobacter aerogenes | | |
| KCTC 2190] | | |
| glycerol dehydrogenase | 65 | WP_017410769.1 |
| [Aeromonas hydrophila] | | |

The recombinant cell comprises one or more genes coding for an enzyme having dihydroxyacetone kinase activity. The dihydroxyacetone kinase enzyme catalyzes at least one of the following reactions:

EC 2.7.1.28: ATP+D-glyceraldehyde<=>ADP+D-glyceraldehyde 3-phosphate (II)

or

EC 2.7.1.29 ATP+glycerone<=>ADP+glycerone phosphate (III)

This family consists of examples of the single chain form of dihydroxyacetone kinase (also called glycerone kinase) that uses ATP (EC 2.7.1.29 or EC 2.7.1.28) as the phosphate donor, rather than a phosphoprotein as in Escherichia coli. This form has separable domains homologous to the K and L subunits of the E. coli enzyme, and is found in yeasts and other eukaryotes and in some bacteria, including Citrobacter freundii. The member from tomato has been shown to phosphorylate dihydroxyacetone, 3,4-dihydroxy-2-butanone, and some other aldoses and ketoses. Members from mammals have been shown to catalyse both the phosphorylation of dihydroxyacetone and the splitting of ribonucleoside diphosphate-X compounds among which FAD is the best substrate. In yeast there are two isozymes of dihydroxyacetone kinase (Dak1 and Dak2). When the cell is a yeast cell the endogenous Dak proteins are preferred according to the invention, in an embodiment they are overexpressed in yeast cell.

The enzyme having dihydroxy acetone kinase activity may be encoded by an endogenous gene, e.g. a DAK1, which endogenous gene is preferably placed under control of a constitutive promoter. The recombinant cell may comprise a genetic modification that increases the specific activity of dihydroxyacetone kinase in the cell.

In an embodiment the recombinant cell comprises one or more nucleic acid sequences encoding a dihydroxy acetone kinase represented by amino acid sequence according to SEQ ID NO: 4, 19, 20 or 21, or by a functional homologue thereof having a sequence identity of at least 50%, preferably at least 60%, 70%, 75%, 80%. 85%, 90% or 95%, which gene is preferably placed under control of a constitutive promoter.

Examples of suitable dihydroxyacetone kinases are listed in table 2(a) to 2(d). At the top of each table the dihydroxyacetone kinase that is BLASTED is mentioned.

TABLE 2(a)
BLAST Query - DAK1 from
Saccharomyces cerevisiae (SEQ ID NO: 4)

| | Identity | |
|---|---|---|
| Description | (%) | Accession number |
| Dak1p [Saccharomyces cerevisiae | 100 | NP_013641.1 |
| S288c] | | |
| dihydroxyacetone kinase | 99 | EDN64325.1 |
| [Saccharomyces cerevisiae YJM789] | | |
| DAK1-like protein | 95 | EJT44075.1 |
| [Saccharomyces kudriavzevii IFO 1802] | | |
| ZYBA0S11-03576g1_1 | 77 | CDF91470.1 |
| [Zygosaccharomyces bailii CLIB 213] | | |
| hypothetical protein | 70 | XP_451751.1 |
| [Kluyveromyces lactis NRRL Y-1140] | | |
| hypothetical protein | 63 | XP_449263.1 |
| [Candida glabrata CBS 138] | | |
| Dak2p [Saccharomyces cerevisiae | 44 | NP_116602.1 |
| S288c] | | |

TABLE 2(b)
BLAST Query - dhaK from Klebsiella pneumoniae (SEQ ID NO: 19)

| Description | Identity (%) | Accession number |
|---|---|---|
| dihydroxyacetone kinase subunit | 100 | YP_002236493.1 |
| DhaK [Klebsiella pneumoniae 342] | | |
| dihydroxyacetone kinase subunit | 99 | WP_004149886.1 |
| K [Klebsiella pneumoniae] | | |
| dihydroxyacetone kinase subunit | 96 | WP_020077889.1 |
| K [Enterobacter aerogenes] | | |
| dihydroxyacetone kinase subunit | 88 | YP_002407536.1 |
| DhaK [Escherichia coli IAI39] | | |
| dihydroxyacetone kinase, DhaK | 87 | WP_001398949.1 |
| subunit [Escherichia coli] | | |

TABLE 2(c)
BLAST Query - DAK1 from Yarrowia lipolytica (SEQ ID NO: 20)

| | Identity | |
|---|---|---|
| Description | (%) | Accession number |
| YALI0F09273p [Yarrowia lipolytica] | 100 | XP_505199.1 |
| dihydroxyacetone kinase | 46 | AAC83220.1 |
| [Schizosaccharomyces pombe] | | |
| dihydroxyacetone kinase Dak1 | 45 | NP_593241.1 |
| [Schizosaccharomyces pombe | | |
| 972h-] | | |
| dihydroxyacetone kinase | 44 | EDV12567.1 |
| [Saccharomyces cerevisiae RM11-1a] | | |
| Dak2p [Saccharomyces cerevisiae | 44 | EEU04233.1 |
| JAY291] | | |
| BN860_19306g1_1 | 44 | CDF87998.1 |
| [Zygosaccharomyces bailii CLIB 213] | | |
| Dak1p [Saccharomyces cerevisiae | 42 | EIW08612.1 |
| CEN.PK113-7D] | | |

TABLE 2(d)
BLAST Query - DAK1 from
Schizosaccharomyces pombe (SEQ ID NO: 21)

| Description | Identity (%) | Accession number |
|---|---|---|
| dihydroxyacetone kinase Dak1 | 100 | NP_593241.1 |
| [Schizosaccharomyces pombe 972h-] | | |
| putative dihydroxyacetone kinase | 48 | EMR88164.1 |
| protein [Botryotinia fuckeliana | | |
| BcDW1] | | |
| Dihydroxyacetone kinase 1 | 48 | ENH64704.1 |
| [Fusarium oxysporum f. sp. cubense | | |
| race 1] | | |
| Dak1p [Saccharomyces cerevisiae | 46 | EIW08612.1 |
| CEN.PK113-7D] | | |
| Dak2p [Saccharomyces cerevisiae | 44 | EEU04233.1 |
| JAY291] | | |
| dihydroxyacetone kinase [Exophiala | 42 | EHY55064.1 |
| dermatitidis NIH/UT8656] | | |

The recombinant cell comprises one or more genes coding for an enzyme in an acetyl-CoA-production pathway. In an embodiment, the one or more genes coding for an enzyme in an acetyl-CoA-production pathway comprises:
one or more genes coding for an enzyme having phosphoketolase (PKL) activity (EC 4.1.2.9 or EC 4.1.2.22); and/or
one or more genes coding for an enzyme having phosphotransacetylase (PTA) activity (EC 2.3.1.8); and or
one or more genes coding for an enzyme having acetate kinase (ACK) activity (EC 2.7.2.12).

The recombinant cell may comprise one or more (heterologous) genes coding for an enzyme having phosphoketolase activity. As used herein, a phosphoketolase catalyzes at least the conversion of D-xylulose 5-phosphate to D-glyceraldehyde 3-phosphate and acetyl phosphate. The phosphoketolase is involved in at least one of the following the reactions:

### EC 4.1.2.9:

D-xylulose-5-phosphate+phosphate acetyl phosphate+D-glyceraldehyde 3-phosphate+H₂O (IV)

D-ribulose-5-phosphate+phosphate acetyl phosphate+D-glyceraldehyde 3-phosphate+H₂O (V)

### EC 4.1.2.22:

D-fructose 6-phosphate+phosphate acetyl phosphate+D-erythrose 4-phosphate+H₂O (VI)

A suitable enzymatic assay to measure phosphoketolase activity is described e.g. in Sonderegger et al. (2004, Applied & Environmental Microbiology, 70(5), pp. 2892-2897). In an embodiment the one or more genes coding for an enzyme having phosphoketolase activity encodes an enzyme having an amino acid sequence according to SEQ ID NO: 5, 6, 7 or 8, or a functional homologue thereof having a sequence identity of at least 50%, preferably at least 60%, 70%, 75%, 80%. 85%, 90% or 95%. Suitable nucleic acid sequences coding for an enzyme having phosphoketolase may in be found in an organism selected from the group of Aspergillus niger, Neurospora crassa, L. casei, L. plantarum, L. plantarum, B. adolescentis, B. bifidum, B. gallicum, B. animalis, B. lactis, L. pentosum, L. acidophilus, P. chrysogenum, A. nidulans, A. clavatus, L. mesenteroides, and O. oenii.

The recombinant cell may comprise one or more (heterologous) genes coding for an enzyme having phosphotransacetylase activity. As used herein, a phosphotransacetylase catalyzes at least the conversion of acetyl phosphate to acetyl-CoA. In an embodiment the one or more genes coding for an enzyme having phosphotransacetylase activity encodes an enzyme having an amino acid sequence according to SEQ ID NO: 9, 10, 11 or 12, or functional homologues thereof having a sequence identity of at least 50% preferably at least 60%, 70%, 75%, 80%. 85%, 90% or 95%. Suitable nucleic acid sequences coding for an enzyme having phosphotransacetylase may in be found in an organism selected from the group of B. adolescentis, B. subtilis, C. cellulolyticum, C. phytofermentans, B. bifidum, B. animalis, L. mesenteroides, Lactobacillus plantarum, M. thermophila, and O. oeniis.

The recombinant cell may comprise one or more (heterologous) genes coding for an enzyme having one or more genes coding for an enzyme having acetate kinase activity (EC 2.7.2.12). Said one or more endogenous genes may encode an acetate kinase having an amino acid sequence according to SEQ ID NO: 1 or 2, or functional homologues thereof having a sequence identity of at least 50%, preferably at least 60%, 70%, 75%, 80%. 85%, 90% or 95%. As used herein, an acetate kinase catalyzes at least the conversion of acetate to acetyl phosphate.

In an embodiment the recombinant cell comprises one or more genes coding for a glycerol transporter. Glycerol that is externally available in the medium (e.g. from the backset in corn mash) or secreted after internal cellular synthesis may be transported into the cell and converted to ethanol by the concomittant (over)expression of a glycerol dehydrogenase and dihydroxy acetone kinase. In an embodiment the recombinant cell comprises one or more genes encoding a heterologous glycerol transporter represented by SEQ ID NO: 13 or 14, or a functional homologue thereof having a sequence identity of at least 60%, preferably at least 70%, 75%, 80%. 85%, 90% or 95%.

Glycerol, a main product of yeast metabolism, is a precursor for several cellular compounds and a regulator of various different metabolic pathways. Some studies suggest that glycerol metabolism appeared very early in the evolutionary process (Weber, 1987). The pathways in which glycerol is involved have been preserved throughout evolution, demonstrating their fundamental importance.

Glycerol is an important substrate in several species' energy metabolism. For instance, glycerol is a precursor involved in lipid synthesis (see e.g. Holms, 1996, FEMS Microbiol. Rev. 21: 85-116, and references therein) and plays an important role in the balance of cell redox potential and inorganic phosphate recycling (see e.g. Ansell et al., 1997, EMBO J. 16: 2179-2187; Alonso-Monge et al., 2003, Eukaryot. Cell 2: 351-361). In prolonged fasting, glycerol can be used as the only source for gluconeogenesis (Baba et al., 1995, Nutrition 11:149-153). In eukaryotic microorganisms it is the main compatible solute produced to counterbalance the low water availability in high-osmotic stressed environments (see e.g. Rep et al., 1999, Microbiol. 145: 715-727; Wang et al., 2001, Biothec. Adv. 19:201-223).

For many years, glycerol was considered to be a lipo-soluble molecule, able to cross cell membranes by simple diffusion (Gancedo et al., 1968, Eur. J. Biochem. 5: 165-172). Yet, this was not consistent with the fact that yeasts retain and accumulate glycerol inside the cell (Blomberg and Adler, 1989, J. Bacteriol. 171: 1087-1092). Indeed, nowadays glycerol transporters (such as channels, facilitators and symporters) have been identified, characterized biochemically and the corresponding genes have been cloned (Neves, 2004, Thesis Universidade do Minho. Departamento de Biologia. Braga, Portugal, and references therein).

Under aerobic conditions, S. cerevisiae is able to utilize glycerol as a sole carbon and energy source. Glycerol degradation is a two-step process; the first step of glycerol phosphorylation occurs in the cytosol, then glycerol-3-phosphate enters the mitochondrion where the second step of conversion to dihydroxyacetone is catalyzed. Dihydroxyacetone is then returned to the cytosol where it enters into either glycolysis or gluconeogenesis. The genes encoding the enzymes catalyzing aerobic glycerol catabolism, GUT1 and GUT2, are carbon source-regulated. Gene expression is repressed when cells are grown on fermentable carbon sources such as glucose and up-regulated on non-fermentable carbon sources such as glycerol or ethanol (see e.g. Grauslund 1999, Nucleic Acids Res 27(22); 4391-4398; Grauslund and Ronnow, 2000, Can J Microbiol 46(12);1096-1100, and references therein). On non-fermentable carbon sources, GUT1 transcription is induced by the transcriptional activators Adr1p, Ino2p and Ino4p, while GUT2 regulation requires the protein kinase Snf1p and the transcriptional activating Hap2p/Hap3p/Hap4p/Hap5p complex. Conversely, the negative regulator Opi1p facilitates GUT1 and GUT2 repression (Grauslund 1999, Nucleic Acids Res 27(22);4391-4398; Grauslund and Ronnow, 2000, Can J Microbiol 46(12);1096-1100).

When the yeast Saccharomyces cerevisiae is grown under anaerobic conditions, glycerol is, after ethanol and carbon dioxide, the most abundant by-product. Glycerol is produced by reduction of the glycolytic intermediate dihydroxyacetone phosphate (DHAP) to glycerol-3-phosphate using NADH as a co-factor, followed by dephosphorylation.

The fermentative pathway from glucose-6-phosphate to ethanol is redox neutral. However, excess NADH is formed in connection with biomass and metabolite synthesis, and this NADH has to be reoxidized. As a consequence, the NADH coupled reduction of DHAP to glycerol serves as a central means of maintaining the redox balance during anaerobic growth (Ansell et al., 1997, EMBO J. 16: 2179-2187).

There are however also other conditions under which yeast produces glycerol. For instance, when S. cerevisiae is exposed to salt stress, the organism responds by increasing the internal concentration of glycerol. The accumulated glycerol functions as an osmolyte, preventing loss of turgor pressure of the cell (Blomberg and Adler 1992).

In SGD (Saccharomyces Genome database; (www.yeastgenome.org) a list of genes that play a role in the synthesis and degradation/metabolism of glycerol can be searched for. In Table 3, the genes that are known to be involved in glycerol metabolism in S. cerevisiae to date, are listed below.

TABLE 3
Genes associated with glycerol metabolism in the yeast S. cerevisiae, and the GO
terms and synonyms these genes. Source: www.yeastgenome.org.

| GO terms | GO synonyms | Associated gene(s) |
|---|---|---|
| glycerol transport | | FPS1; GUP1; GUP2; STL1 |
| glycerol catabolic process | glycerol breakdown; | DAK1; DAK2; GUP1; GUT1; |
| | glycerol catabolism; | GUT2 |
| | glycerol degradation | |
| glycerol biosynthetic process | glycerol anabolism; | HOR2; RHR2; YIG1 |
| | glycerol biosynthesis; | |
| | glycerol formation; | |
| | glycerol synthesis | |
| anaerobic glycerol catabolic process | glycerol fermentation | DAK1; DAK2 |
| glycerol metabolic process | glycerol metabolism | DAK1; DAK2; DGA1; FPS1 |
| | | GDE1; GPD2; GUT1; GUT2; |
| | | PGC1; TCO89 |
| glycerol-3-phosphate transport | | GIT1; PHO91 |
| intracellular accumulation of glycerol | | GPD1 |
| glycerol-3-phosphate metabolic | glycerol-3-phosphate metabolism | GPD1; GPD2; GUT1; GUT2 |
| process | | |
| glycerol ether metabolic process | glycerol ether metabolism | MPD1; PDI1; TRX1; TRX2; |
| | | TRX3 |
| glycerol-3-phosphate catabolic process | glycerol-3-phosphate breakdown; | GPD1; GPD2 |
| | glycerol-3-phosphate catabolism; | |
| | glycerol-3-phosphate degradation | |
| positive regulation of glyceroltransport | | ASK10; RGC1 |
| MAPK cascade involved in | High Osmolarity Glycerol (HOG) MAPK | CDC37; SSK2; SSK22; |
| osmosensory signaling pathway | pathway; | STE11; STE50 |
| | Hog1 MAPK pathway; | |
| | MAPKKK cascade during osmolarity | |
| | sensing; | |
| | MAPKKK cascade involved in | |
| | osmosensory signaling pathway; | |
| | MAPKKK cascade involved in | |
| | osmosensory signaling pathway; | |
| | osmolarity sensing, MAPKKK cascade | |

Plasma membrane proteins play pivotal roles in all cellular functions. The plasma membrane which encompasses the cytosol of each cell allows the microbe to maintain fairly constant intracellular conditions. The membrane enables the cell to selectively take up exogenous nutrients from the environment and to excrete certain solutes from the cytosol into the cell's surroundings. Although some substances, such as for instance water and ethanol, diffuse readily through membranes, solutes are generally taken across the membranes by enzyme-like carriers (also called ‘permeases’ or ‘transport systems’).

The transport of solutes by primary active transporters is energy-driven in the first place, such as by energy supplied from ATP hydrolysis, photon absorption, electron flow, substrate decarboxylation, or methyl transfer. If charged molecules are pumped in one direction as a consequence of the consumption of a primary cellular energy source, an electrochemical potential is the result. The resulting chemiosmotic gradient can then be used to drive the transport of additional molecules via secondary carrier structures which just facilitate the transport of one or more molecules across the membrane.

The last two decades the existence of a multitude of previously unknown protein families of primary and secondary transporters has been clarified by the emergence of genomics strategies and making use of the many performed biochemical and molecular genetics studies. The two main transporter families of which proteins were found throughout all living organism are of the ATP-binding cassette (ABC) superfamily and the major facilitator superfamily (MFS), also known as the uniporter-symporter-antiporter family. Whereas ABC family permeases consist of multiple components and are primary active transporters, capable of transporting both small molecules and macromolecules only after generating energy through ATP hydrolysis, the MFS transporters consist of a single polypeptide of a secondary carrier which facilitates transport of small solutes in response to a chemiosmotic ion gradient. ABC superfamily and MFS proteins account for almost half of the solute transporters encoded within the microbe genomes (reviewed by Pao et al, 1998, Microbiol Mol Biol Rev.; 62 pp.1-34, and Saier et al, 1999, J Mol Microbiol Biotechnol, 1 pp.257-279). Also, channels exist, such as e.g. aquaporin (AQP) water channels that facilitate rapid water or solute transport across either the plasma or vacuolar membranes (Coury et al 1999, J. Bacteriol. vol. 181, NO: 14, p4437-4440).

In case of S. cerevisiae, four different genes have been implicated with glycerol transport (see Table 4): FPS1, GUP1, GUP2 and STL1. The following gene descriptions have been assigned to these genes (www.yeastgenome.org and references therein).

TABLE 4
Description of protein function of proteins encoded by FPS1, GUP1, GUP2 and STL1.

| Gene name | |
|---|---|
| (alias) | Description |
| FPS1 | Aquaglyceroporin, plasma membrane channel; involved in efflux of glycerol and |
| (YLL043w) | xylitol, and in uptake of acetic acid and the trivalent metalloids arsenite and |
| | antimonite; role in mediating passive diffusion of glycerol is key factor in |
| | maintenance of redox balance; member of major intrinsic protein (MIP) family; |
| | phosphorylated by Hog1p MAPK under acetate stress; deletion improves xylose |
| | fermentation |
| GUP1 | Plasma membrane protein involved in remodeling GPI anchors; member of the |
| (YGL084c) | MBOAT family of putative membrane-bound O-acyltransferases; proposed to be |
| | involved in glycerol transport; GUP1 has a paralog, GUP2, that arose from the |
| | whole genome duplication |
| GUP2 | Probable membrane protein; possible role in proton symport of glycerol; member |
| (YPL189w) | of the MBOAT family of putative membrane-bound O-acyltransferases; GUP2 |
| | has a paralog, GUP1, that arose from the whole genome duplication |
| STL1 | Glycerol proton symporter of the plasma membrane, subject to glucose-induced |
| (YDR536w) | inactivation, strongly but transiently induced when cells are subjected to osmotic |
| | shock |

For a number of reasons, overexpression of one of these four S. cerevisiae membrane proteins is not expected to facilitate the transport of glycerol across the plasma membrane under fermentation conditions. FPS1, GUP1 and GUP2 do not play a role in the uptake of glycerol. STL1 encodes a glycerol transporter, but is subject to repression at the transcription level and glucose-inactivation at the protein level (Table 4). Two proteins were selected, heterologous to S. cerevisiae, implicated in glycerol transport. These putative glycerol transporters, either being a facilitator, a channel, a uniporter or a symporter, are herein shown, upon overexpression in strains having anaerobic glycerol conversion pathway (comprised of a glycerol dehydrogenase and a dihydroxyacetone kinase), an acetyl-CoA production pathway, and an acetylating NAD+-dependant acetaldehyde dehydrogenase to result in an increase in the conversion of glycerol, and subsequently into ethanol, due to improved glycerol transporting activity in said yeast cells. Ideally, the transporter is not repressed or inactivated by glucose.

The selected glycerol transporters are listed in Table 5.

TABLE 5
Selected glycerol transporter genes

| Species | Gene Name | # AA | Protein Sequence |
|---|---|---|---|
| Danio rerio | AQP9 | 291 | SEQ ID NO: 13 |
| | NP_001171215 | | |
| Zygosaccharomyces rouxii | ZYRO0E01210p | 592 | SEQ ID NO: 14 |

BLAST identity searches (protein) for the above glycerol transporters are given below in table 6 a) and 6 b) and indicate other glycerol transporters that are suitable for use in cells of the invention.

TABLE 6 (a)
BLAST Query - AQP9 (NP_001171215) from Danio rerio

| | Identity | Accession |
|---|---|---|
| Description | (%) | number |
| aquaporin-9 [Danio rerio] >gb\|ACB10576.1\| | 100 | NP_001171215.1 |
| aquaporin-9b [Danio rerio] | | |
| aquaglyceroporin [Osmerus mordax] | 73 | ABG24574.1 |
| PREDICTED: aquaporin-9 [Gorilla gorilla | 51 | XP_004056310.1 |
| gorilla] | | |
| aquaporin 3 (Gill blood group) [Xenopus | 52 | NP_001081876.1 |
| laevis] >emb\|CAA10517.1\|aquaporin-3 | | |
| [Xenopus laevis] | | |

TABLE 6 e)
BLAST Query - ZYRO0E01210p from Zygosaccharomyces rouxii

| Description | Identity (%) | Accession number |
|---|---|---|
| ZYRO0E01210p [Zygosaccharomyces rouxii] | 100 | XP_002498999.1 |
| >emb\|CAR30744.1\| ZYRO0E01210p [Zygosaccharomyces | | |
| rouxii] | | |
| BN860_18536g1_1 [Zygosaccharomyces bailii CLIB 213] | 82 | CDF87965.1 |
| hypothetical protein TDEL_0B07220 [Torulaspora delbrueckii] | 66 | XP_003680062.1 |
| >emb\|CCE90851.1\| hypothetical protein TDEL_0B07220 | | |
| [Torulaspora delbrueckii] | | |
| Stl1p [Saccharomyces cerevisiae S288c] >sp\|P39932.2 | 66 | NP_010825.3 |
| sugar transporter STL1 [Candida albicans WO-1] | 64 | EEQ46634.1 |
| monosaccharide transporter [Cryptococcus gattii WM276] | 45 | XP_003193210.1 |
| >gb\|ADV21423.1 | | |

In an embodiment the recombinant cell comprises a deletion or disruption of one or more endogenous nucleotide sequences encoding a glycerol exporter. In S. cerevisiae, one such a glycerol exporter is encoded by FPS1 (see Table 3 and 4).

In an embodiment the recombinant cell either lacks enzymatic activity needed for NADH-dependent glycerol synthesis or has reduced enzymatic activity needed for NADH-dependent glycerol synthesis compared to its corresponding wild type (yeast) cell. Alternatively, strains that are defective in glycerol production may be obtained by random mutagenesis followed by selection of strains with reduced or absent activity of GPD and/or GPP.

In an embodiment the recombinant cell comprises a deletion or disruption of one or more endogenous nucleotide sequences encoding a glycerol 3-phosphate phosphohydrolase, such as S. cerevisiae GPP1 or GPP2 Such a deletion or disruption may result in decrease or removal of enzymatic activity. As used herein, a glycerol 3-phosphate phosphohydrolase catalyzes at least the following reaction:

glycerol phosphate glycerol+phosphate (VII)

In an embodiment the recombinant cell comprises a deletion or disruption of one or more endogenous nucleotide sequences encoding a glycerol-3-phosphate dehydrogenase. Such a deletion or disruption may result in decrease or removal of enzymatic activity. As used herein, a glycerol 3-phosphate dehydrogenase catalyzes at least the following reaction:

dihydroxyacetone phosphate+NADH glycerol phosphate+NAD⁺ (VIII)

Glycerol-3-phosphate dehydrogenase may be entirely deleted, or at least a part is deleted which encodes a part of the enzyme that is essential for its activity. In particular, good results have been achieved with a S. cerevisiae cell, wherein the open reading frames of the GPD1 gene and of the GPD2 gene have been inactivated. Inactivation of a structural gene (target gene) can be accomplished by a person skilled in the art by synthetically synthesizing or otherwise constructing a DNA fragment consisting of a selectable marker gene flanked by DNA sequences that are identical to sequences that flank the region of the host cell's genome that is to be deleted. In particular, good results have been obtained with the inactivation of the GPD1 and GPD2 genes in Saccharomyces cerevisiae by integration of the marker genes kanMX and hphMX4. Subsequently this DNA fragment is transformed into a host cell. Transformed cells that express the dominant marker gene are checked for correct replacement of the region that was designed to be deleted, for example by a diagnostic polymerase chain reaction or Southern hybridization. The deleted or disrupted glycerol-3-phosphate dehydrogenase preferably belongs to EC 1.1.5.3, such as GUT2, or to EC 1.1.1.8, such as GPD1 and or GPD2. In embodiment the cell is free of genes encoding NADH-dependent glycerol-3-phosphate dehydrogenase.

In an embodiment the recombinant cell either lacks enzymatic activity needed for the production of glycerol 3-phosphate or has reduced enzymatic activity needed for the production of glycerol 3-phosphate compared to its corresponding wild type (yeast) cell. The recombinant cell may comprise a deletion or disruption of one or more endogenous nucleotide sequences encoding a glycerol kinase (EC 2.7.1.30). An example of such an enzyme is Gut1p. As used herein, a glycerol kinase catalyzes at least the following reaction:

glycerol+phosphate 4 glycerol 3-phosphate (IX)

In an embodiment the recombinant cell either lacks enzymatic activity needed for the production of acetic acid from acetaldehyde or has reduced enzymatic activity needed for the production of acetic acid from acetaldehyde compared to its corresponding wild type (yeast) cell. The recombinant cell may comprise a deletion or disruption of one or more endogenous genes encoding an enzyme having NAD(P)H dependent aldehyde dehydrogenase activity (EC 1.2.1.4). One such an aldehyde dehydrogenase is encoded by S. cerevisiae ALD6. As used herein, an aldehyde dehydrogenase catalyzes at least the following reaction:

acetaldehyde+NAD(P)⁺<->acetic acid+NAD(P)H (X)

The recombinant cell comprises one or more genes coding for an enzyme having at least NAD⁻ dependent acetylating acetaldehyde dehydrogenase activity. As used herin, an NAD⁺ dependent acetylating acetaldehyde dehydrogenase catalyses at least the conversion of acetyl-CoA to acetaldehyde. This conversion can be represented by the equilibrium reaction formula:

acetyl-CoA+NADH+H⁺<->acetaldehyde+NAD+CoA (XI)

In an embodiment the one or more genes encoding an enzyme having at least NAD+ dependent acetylating acetaldehyde dehydrogenase activity encodes an enzyme having an amino acid sequence according to SEQ ID NO: 3, 22, 23, 24 or 25, or a functional homologue thereof having a sequence identity of at least 50%, preferably at least 60%, 70%, 75%, 80%. 85%, 90% or 95%. Said NAD⁺ dependent acetylating acetaldehyde dehydrogenase may catalyse the reversible conversion of acetyl-Coenzyme-A to acetaldehyde and the subsequent reversible conversion of acetaldehyde to ethanol, which enzyme may comprise both NAD⁺ dependent acetylating acetaldehyde dehydrogenase (EC 1.2.1.10 or EC 1.1.1.2) activity and NAD⁺ dependent alcohol dehydrogenase activity (EC 1.1.1.1). Thus, this enzyme allows the re-oxidation of NADH when acetyl-CoA is generated from acetate present in the growth medium, and thereby glycerol synthesis is no longer needed for redox cofactor balancing. The nucleic acid sequence encoding the NAD⁺ dependent acetylating acetaldehyde dehydrogenase may in principle originate from any organism comprising a nucleic acid sequence encoding said dehydrogenase. Known NAD+ dependent acetylating acetaldehyde dehydrogenases that can catalyse the NADH-dependent reduction of acetyl-Coenzyme A to acetaldehyde may in general be divided in three types of NAD⁺ dependent acetylating acetaldehyde dehydrogenase functional homologues:

1) Bifunctional proteins that catalyse the reversible conversion of acetyl-CoA to acetaldehyde, and the subsequent reversible conversion of acetaldehyde to ethanol. An example of this type of proteins is the AdhE protein in E. coli (Gen Bank No: NP_415757). AdhE appears to be the evolutionary product of a gene fusion. The NH2— terminal region of the AdhE protein is highly homologous to aldehyde:NAD+ oxidoreductases, whereas the COOH-terminal region is homologous to a family of Fe²⁺ dependent ethanol:NAD+ oxidoreductases (Membrillo-Hernandez et al., (2000) J. Biol. Chem. 275: 33869-33875). The E. coli AdhE is subject to metal-catalyzed oxidation and therefore oxygen-sensitive (Tamarit et al. (1998) J. Biol. Chem. 273:3027-32).

2) Proteins that catalyse the reversible conversion of acetyl-Coenzyme A to acetaldehyde in strictly or facultative anaerobic micro-organisms but do not possess alcohol dehydrogenase activity. An example of this type of proteins has been reported in Clostridium kluyveri (Smith et al. (1980) Arch. Biochem. Biophys. 203: 663-675). An acetylating acetaldehyde dehydrogenase has been annotated in the genome of Clostridium kluyveri DSM 555 (GenBank No: EDK33116). A homologous protein AcdH is identified in the genome of Lactobacillus plantarum (GenBank No: NP_784141). Another example of this type of proteins is the said gene product in Clostridium beijerinckii NRRL B593 (Toth et al. (1999) Appl. Environ. Microbiol. 65: 4973-4980, GenBank No: AAD31841).

3) Proteins that are part of a bifunctional aldolase-dehydrogenase complex involved in 4-hydroxy-2-ketovalerate catabolism. Such bifunctional enzymes catalyze the final two steps of the meta-cleavage pathway for catechol, an intermediate in many bacterial species in the degradation of phenols, toluates, naphthalene, biphenyls and other aromatic compounds (Powlowski and Shingler (1994) Biodegradation 5, 219-236). 4-Hydroxy-2-ketovalerate is first converted by 4-hydroxy-2-ketovalerate aldolase to pyruvate and acetaldehyde, subsequently acetaldehyde is converted by acetylating acetaldehyde dehydrogenase to acetyl-CoA. An example of this type of acetylating acetaldehyde dehydrogenase is the DmpF protein in Pseudomonas sp CF600 (GenBank No: CAA43226) (Shingler et al. (1992) J. Bacteriol. 174:711-24). The E. coli MphF protein (Ferrandez et al. (1997) J. Bacteriol. 179: 2573-2581, GenBank No: NP_{—} 414885) is homologous to the DmpF protein in Pseudomonas sp. CF600.

A suitable nucleic acid sequence may in particular be found in an organism selected from the group of Escherichia, in particular E. coli; Mycobacterium, in particular Mycobacterium marinum, Mycobacterium ulcerans, Mycobacterium tuberculosis; Carboxydothermus, in particular Carboxydothermus hydrogenoformans; Entamoeba, in particular Entamoeba histolytica; Shigella, in particular Shigella sonnei; Burkholderia, in particular Burkholderia pseudo mallei, Klebsiella, in particular Klebsiella pneumoniae; Azotobacter, in particular Azotobacter vinelandii; Azoarcus sp; Cupriavidus, in particular Cupriavidus taiwanensis; Pseudomonas, in particular Pseudomonas sp. CF600; Pelomaculum, in particular Pelotomaculum thermopropionicum. Preferably, the nucleic acid sequence encoding the NAD⁺ dependent acetylating acetaldehyde dehydrogenase originates from Escherichia, more preferably from E. coli.

Particularly suitable is an mhpF gene from E. coli, or a functional homologue thereof. This gene is described in Fernindez et al. (1997) J. Bacteriol. 179:2573-2581. Good results have been obtained with S. cerevisiae, wherein an mhpF gene from E. coli has been incorporated. In a further advantageous embodiment the nucleic acid sequence encoding an (acetylating) acetaldehyde dehydrogenase is from Pseudomonas, in particular dmpF, e.g. from Pseudomonas sp. CF600.

The nucleic acid sequence encoding the NAD⁺ dependent, acetylating acetaldehyde dehydrogenase may be a wild type nucleic acid sequence. Further, an acetylating acetaldehyde dehydrogenase (or nucleic acid sequence encoding such activity) may in for instance be selected from the group of Escherichia coli adhE, Entamoeba histolytica adh2, Staphylococcus aureus adhE, Piromyces sp. E2 adhE, Clostridium kluyveri EDK33116, Lactobacillus plantarum acdH, Escherichia coli eutE, Listeria innocua acdH, and Pseudomonas putida YP 001268189. For sequences of some of these enzymes, nucleic acid sequences encoding these enzymes and methodology to incorporate the nucleic acid sequence into a host cell, reference is made to WO2009/013159, in particular Example 3, Table 1 (page 26) and the Sequence ID numbers mentioned therein, of which publication Table 1 and the sequences represented by the Sequence ID numbers mentioned in said Table are incorporated herein by reference.

It is further understood, that in a preferred embodiment, that the cell has endogenous alcohol dehydrogenase activities which allow the cell, being provided with acetaldehyde dehydrogenase activity, to complete the conversion of acetyl-CoA into ethanol. It is further also preferred that the host cell has endogenous acetyl-CoA synthetase which allow the cell, being provided with acetaldehyde dehydrogenase activity, to complete the conversion of acetic acid (via acetyl-CoA) into ethanol.

Examples of suitable enzymes are adhE of Escherichia coli, acdH of Lactobacillus plantarum, eutE of Escherichia coli, Lin1129 of Listeria innocua and adhE from Staphylococcus aureus. See below tables 7(a) to 7(e) for BLAST of these enzymes, giving suitable alternative alcohol/acetaldehyde dehydrogenases that are tested in the examples below.

TABLE 7(a)
BLAST Query - adHE from Escherichia coli

| | Identity | |
|---|---|---|
| Description | (%) | Accession number |
| bifunctional acetaldehyde-CoA/alcohol | 100 | NP_309768.1 |
| dehydrogenase [Escherichia coli | | |
| O157:H7 str. Sakai] | | |
| bifunctional acetaldehyde-CoA/alcohol | 99 | YP_540449.1 |
| dehydrogenase [Escherichia coli | | |
| UTI89] | | |
| bifunctional acetaldehyde-CoA/alcohol | 95 | YP_001177024.1 |
| dehydrogenase [Enterobacter | | |
| sp. 638] | | |

TABLE 7(b)
BLAST Query - acdH from Lactobacillus plantarum

| | Identity | |
|---|---|---|
| Description | (%) | Accession number |
| acetaldehyde dehydrogenase | 100 | YP_004888365.1 |
| [Lactobacillus plantarum WCFS1] | | |
| acetaldehyde dehydrogenase | 95 | CCC16763.1 |
| [Lactobacillus pentosus IG1] | | |
| aldehyde-alcohol dehydrogenase | 58 | WP_016251441.1 |
| [Enterococcus cecorum] | | |
| aldehyde-alcohol dehydrogenase 2 | 57 | WP_016623694.1 |
| [Enterococcus faecalis] | | |
| bifunctional acetaldehyde-CoA/alcohol | 55 | WP_010493695.1 |
| dehydrogenase [Lactobacillus zeae] | | |
| alcohol dehydrogenase | 54 | WP_003280110.1 |
| [Bacillus thuringiensis] | | |
| bifunctional acetaldehyde-CoA/alcohol | 53 | WP_009931954.1 |
| dehydrogenase, partial | | |
| [Listeria monocytogenes] | | |

TABLE 7(c)
BLAST Query - eutE from Escherichia coli

| | Identity | Accession |
|---|---|---|
| Description | (%) | number |
| aldehyde oxidoreductase, ethanolamine | 100 | NP_416950.1 |
| utilization protein [Escherichia coli | | |
| str. K-12 substr. MG1655] | | |
| ethanolamine utilization; acetaldehyde | 99 | NP_289007.1 |
| dehydrogenase [Escherichia coli | | |
| O157:H7 str. EDL933] | | |
| aldehyde dehydrogenase [Escherichia | 99 | WP_001075674.1 |
| albertii] | | |

TABLE 7(d)
BLAST Query - Lin1129 from Listeria innocua

| | Identity | |
|---|---|---|
| Description | (%) | Accession number |
| aldehyde dehydrogenase [Listeria | 100 | NP_470466.1 |
| innocua] >emb\|CAC96360.1\| lin1 129 | | |
| [Listeria innocua Clip11262] | | |
| ethanolamine utilization protein EutE | 99 | WP_003761764.1 |
| [Listeria innocua] aldehyde | 95 | AGR09081.1 |
| dehydrogenase [Listeria monocytogenes] | | |
| hypothetical protein [Enterococcus | 64 | WP_010739890.1 |
| malodoratus] | | |
| aldehyde dehydrogenase [Yersinia | 59 | WP_004699364.1 |
| aldovae] | | |
| aldehyde dehydrogenase EutE | 58 | WP_004205473.1 |
| [Klebsiella pneumoniae] | | |

TABLE 7(e)
BLAST Query - adhE from Staphylococcus aureus

| | Identity | |
|---|---|---|
| Description | (%) | Accession number |
| bifunctional acetaldehyde-CoA/alcohol | 100 | NP_370672.1 |
| dehydrogenase [Staphylococcus aureus | | |
| subsp. aureus Mu50] | | |
| aldehyde dehydrogenase family protein | 99 | YP_008127042.1 |
| [Staphylococcus aureus CA-347] | | |
| bifunctional acetaldehyde-CoA/alcohol | 85 | WP_002495347.1 |
| dehydrogenase [Staphylococcus | | |
| epidermidis] | | |
| aldehyde-alcohol dehydrogenase 2 | 75 | WP_016623694.1 |
| [Enterococcus faecalis] | | |

In an embodiment the cell comprises one or more nucleotide sequence encoding a acetyl-CoA synthetase (E.C. 6.2.1.1);

Acetyl-CoA synthetase (also known as acetate-CoA ligase and acetyl-activating enzyme) is a ubiquitous enzyme, found in both prokaryotes and eukaryotes, which catalyses the formation of acetyl-CoA from acetate, coenzyme A (CoA) and ATP as shown below:

ATP+acetate+CoA=AMP+diphosphate+acetyl-CoA (XII)

The activity of this enzyme is crucial for maintaining the required levels of acetyl-CoA, a key intermediate in many important biosynthetic and catabolic processes. It is especially important in eukayotic species as it is the only route for the activation of acetate to acetyl-CoA in these organisms (some prokaryotic species can also activate acetate by either acetate kinase/phosphotransacetylase or by ADP-forming acetyl-CoA synthase). Eukaryotes typically have two isoforms of acetyl-CoA synthase, a cytosolic form involved in biosynthetic processes and a mitochondrial form primarily involved in energy generation.

The crystal structures of a eukaryotic (e.g. from yeast) and bacterial (e.g. from Salmonella) form of this enzyme have been determined. The yeast enzyme is trimeric, while the bacterial enzyme is monomeric. The trimeric state of the yeast protein may be unique to this organism however, as the residues involved in the trimer interface are poorly conserved in other sequences. Despite differences in the oligomeric state of the two enzyme, the structures of the monomers are almost identical. A large N-terminal domain (˜500 residues) containing two parallel beta sheets is followed by a small (˜110 residues) C-terminal domain containing a three-stranded beta sheet with helices. The active site occurs at the domain interface, with its contents determining the orientation of the C-terminal domain.

When the cell is a yeast cell the endogenous ACS are preferred according to the invention, in an embodiment they are overexpressed in yeast cell.

Examples of suitable are listed in table 8. At the top of table 8 the ACS2 used in the examples and that is BLASTED is mentioned.

TABLE 8
BLAST Query - ACS2 from Saccharomyces cerevisiae

| Description | Identity (%) | Accession number |
|---|---|---|
| acetate--CoA ligase ACS2 [Saccharomyces cerevisiae | 100 | NP_013254.1 |
| S288c] | | |
| acetyl CoA synthetase [Saccharomyces cerevisiae | 99 | EDN59693.1 |
| YJM789] | | |
| acetate--CoA ligase [Kluyveromyces lactis NRRL Y- | 85 | XP_453827.1 |
| 1140] | | |
| acetate--CoA ligase [Candida glabrata CBS 138] | 83 | XP_445089.1 |
| acetate--CoA ligase [Scheffersomyces stipitis CBS 6054] | 68 | XP_001385819.1 |
| acetyl-coenzyme A synthetase FacA [Aspergillus | 63 | EDP50475.1 |
| fumigatus A1163] | | |
| acetate--CoA ligase facA-Penicillium chrysogenum | 62 | XP_002564696.1 |
| [Penicillium chrysogenum Wisconsin 54-1255] | | |

In an embodiment the recombinant cell overexpresses the one or more endogenous or heterologous genes encoding enzyme activities in the non-oxidative pentose phosphate pathway under control of a constitutive promoter. Said enzymatic activities are at least transketolase (EC 2.2.1.1, encoded in S. cerevisiae by TKL1 and TKL2), transaldolase (EC 2.2.1.2, encoded in S. cerevisiae by TAL1 and NQM1), D-ribulose-5-phosphate 3-epimerase (EC 5.1.3.1, encoded in S. cerevisiae by RPE1), ribose-5-phosphate ketol-isomerase (EC 5.3.1.6, encoded in S. cerevisiae by RKI1).

The recombinant cell may contain genes of a pentose metabolic pathway non-native to the cell and/or that allow the recombinant cell to convert pentose(s). In one embodiment, the recombinant cell may comprise one or two or more copies of one or more xylose isomerases and/or one or two or more copies of one or more xylose reductase and xylitol dehydrogenase genes, allowing the recombinant cell to convert xylose. In an embodiment thereof, these genes may be integrated into the recombinant cell genome. In another embodiment, the recombinant cell comprises the genes araA, araB and araD. It is then able to ferment arabinose. In one embodiment of the invention the recombinant cell comprises xylA-gene, XYL1 gene and XYL2 gene and/or XKS1-gene, to allow the recombinant cell to ferment xylose; deletion of the aldose reductase (GRE3) gene; overexpression of one or more PPP-genes, e.g. TAL1, TAL2, TKL1, TKL2, RPE1 and RKI1 to allow the increase of the flux through the pentose phosphate path-way in the cell, and/or overexpression of GAL2 and/or deletion of GAL80. Thus though inclusion of the above genes, suitable pentose or other metabolic pathway(s) may be introduced in the recombinant cell that were non-native in the (wild type) recombinant cell.

In an embodiment, the following genes may be introduced in the recombinant cell by introduction into a host cell:
1) a set consisting of PPP-genes TAL1, TKL1, RPE1 and RKI1, optionally under control of strong constitutive promoter;
2) a set consisting of a xylA-gene under under control of strong constitutive promoter;
3) a set comprising a XKS1-gene under control of strong constitutive promoter,
4) a set consisting of the bacterial genes araA, araB and araD under control of a strong constitutive promoter,
5) deletion of an aldose reductase gene

The above cells may be constructed using known recombinant expression techniques. The co-factor modification may be effected before, simultaneous or after any of the modifications 1-5 above.

The recombinant cell according to the invention may be subjected to evolutionary engineering to improve its properties. Evolutionary engineering processes are known processes. Evolutionary engineering is a process wherein industrially relevant phenotypes of a microorganism, herein the recombinant cell, can be coupled to the specific growth rate and/or the affinity for a nutrient, by a process of rationally set-up natural selection. Evolutionary Engineering is for instance described in detail in Kuijper, M, et al, FEMS, Eukaryotic cell Research 5(2005) 925-934, WO2008041840 and WO2009112472. After the evolutionary engineering the resulting pentose fermenting recombinant cell is isolated. The isolation may be executed in any known manner, e.g. by separation of cells from a recombinant cell broth used in the evolutionary engineering, for instance by taking a cell sample or by filtration or centrifugation.

In an embodiment, the recombinant cell is marker-free. As used herein, the term “marker” refers to a gene encoding a trait or a phenotype which permits the selection of, or the screening for, a host cell containing the marker. Marker-free means that markers are essentially absent in the recombinant cell. Being marker-free is particularly advantageous when antibiotic markers have been used in construction of the recombinant cell and are removed thereafter. Removal of markers may be done using any suitable prior art technique, e.g. intramolecular recombination.

In one embodiment, the recombinant cell is constructed on the basis of an inhibitor tolerant host cell, wherein the construction is conducted as described hereinafter. Inhibitor tolerant host cells may be selected by screening strains for growth on inhibitors containing materials, such as illustrated in Kadar et al, Appl. Biochem. Biotechnol. (2007), Vol. 136-140, 847-858, wherein an inhibitor tolerant S. cerevisiae strain ATCC 26602 was selected.

To increase the likelihood that enzyme activity is expressed at sufficient levels and in active form in the recombinant cell, the nucleotide sequence encoding these enzymes, as well as the Rubisco enzyme and other enzymes of the disclosure are preferably adapted to optimise their codon usage to that of the cell in question.

The adaptiveness of a nucleotide sequence encoding an enzyme to the codon usage of a cell may be expressed as codon adaptation index (CAI). The codon adaptation index is herein defined as a measurement of the relative adaptiveness of the codon usage of a gene towards the codon usage of highly expressed genes in a particular cell or organism. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al., 2003, Nucleic Acids Res. 31(8):2242-51). An adapted nucleotide sequence preferably has a CAI of at least 0.2, 0.3, 0.4, 0.5, 0.6, 0.7, 0.8 or 0.9. Most preferred are the sequences which have been codon optimised for expression in the host cell in question such as e.g. S. cerevisiae cells.

In an embodiment the recombinant cell a yeast cell. Such yeast cell may be selected from Saccharomycetaceae, in particular from the group of Saccharomyces, such as Saccharomyces cerevisiae; Kluyveromyces, such as Kluyveromyces marxianus; Pichia, such as Pichia stipitis or Pichia angusta; Zygosaccharomyces, such as Zygosaccharomyces bailii; and Brettanomyces, such as Brettanomyces intermedius, Issatchenkia, such as Issatchenkia orientalis and Hansenula.

In another embodiment the recombinant cell is a prokaryotic cell, such as selected from the list consisting of Clostridium, Zymomonas, Thermobacter, Escherichia, Lactobacillus, Geobacillus and Bacillus.

The invention further provides the use of a recombinant cell for preparation of ethanol. The invention also provides the use of a recombinant cell for preparation of succinic acid.

The invention further provides a process for preparing fermentation product, comprising preparing a fermentation product from a fermentable carbohydrate, in particular selected from the group of glucose, fructose, sucrose, maltose, xylose, arabinose, galactose and mannose which preparation is carried out under anaerobic conditions using a recombinant cell according to the invention.

In the context of the invention “the fermentable carbohydrate” may be part of a composition. Thus, the present invention includes a process to produce a fermentation product comprising:
fermenting a composition comprising a fermentable carbohydrate, in particular selected from the group of glucose, fructose, sucrose, maltose, xylose, arabinose, galactose and mannose under anaerobic conditions in the presence of a cell according to the invention; and
recovering the fermentation product.

In an embodiment one such composition is a biomass hydrolysate. Such biomass hydrolysate may be a lignocellulosic biomass hydrolysate. Lignocellulose herein includes hemicellulose and hemicellulose parts of biomass. Also lignocellulose includes lignocellulosic fractions of biomass. Suitable lignocellulosic materials may be found in the following list: orchard primings, chaparral, mill waste, urban wood waste, municipal waste, logging waste, forest thinnings, short-rotation woody crops, industrial waste, wheat straw, oat straw, rice straw, barley straw, rye straw, flax straw, soy hulls, rice hulls, rice straw, corn gluten feed, oat hulls, sugar cane, corn stover, corn stalks, corn cobs, corn husks, switch grass, miscanthus, sweet sorghum, canola stems, soybean stems, prairie grass, gamagrass, foxtail; sugar beet pulp, citrus fruit pulp, seed hulls, cellulosic animal wastes, lawn clippings, cotton, seaweed, trees, softwood, hardwood, poplar, pine, shrubs, grasses, wheat, wheat straw, sugar cane bagasse, corn, corn husks, corn hobs, corn kernel, fiber from kernels, products and by-products from wet or dry milling of grains, municipal solid waste, waste paper, yard waste, herbaceous material, agricultural residues, forestry residues, municipal solid waste, waste paper, pulp, paper mill residues, branches, bushes, canes, corn, corn husks, an energy crop, forest, a fruit, a flower, a grain, a grass, a herbaceous crop, a leaf, bark, a needle, a log, a root, a sapling, a shrub, switch grass, a tree, a vegetable, fruit peel, a vine, sugar beet pulp, wheat midlings, oat hulls, hard or soft wood, organic waste material generated from an agricultural process, forestry wood waste, or a combination of any two or more thereof. Lignocellulose, which may be considered as a potential renewable feedstock, generally comprises the polysaccharides cellulose (glucans) and hemicelluloses (xylans, heteroxylans and xyloglucans). In addition, some hemicellulose may be present as glucomannans, for example in wood-derived feedstocks. The enzymatic hydrolysis of these polysaccharides to soluble sugars, including both monomers and multimers, for example glucose, cellobiose, xylose, arabinose, galactose, fructose, mannose, rhamnose, ribose, galacturonic acid, glucuronic acid and other hexoses and pentoses occurs under the action of different enzymes acting in concert. In addition, pectins and other pectic substances such as arabinans may make up considerably proportion of the dry mass of typically cell walls from non-woody plant tissues (about a quarter to half of dry mass may be pectins). Lignocellulosic material may be pretreated. The pretreatment may comprise exposing the lignocellulosic material to an acid, a base, a solvent, heat, a peroxide, ozone, mechanical shredding, grinding, milling or rapid depressurization, or a combination of any two or more thereof. This chemical pretreatment is often combined with heat-pretreatment, e.g. between 150-220° C. for 1 to 30 minutes.

In an embodiment the fermentable carbohydrate is obtained from starch, lignocellulose, and/or pectin.

The starch, lignocellulose, and/or pectin may be contacted with an enzyme composition, wherein one or more sugar is produced, and wherein the produced sugar is fermented to give a fermentation product, wherein the fermentation is conducted with a cell of the invention.

The fermentation product may be one or more of ethanol, butanol, organic acid, lactic acid, a plastic, an organic acid, a solvent, an animal feed supplement, a pharmaceutical, a vitamin, an amino acid, an enzyme or a chemical feedstock.

The process is particularly useful when glycerol is fed externally to the process, such as crude glycerol from transesterification-based biodiesel production or recirculation of backset, which is then taken up and converted to ethanol by the recombinant cell.

In an embodiment the composition comprises an amount of undissociated acetic acid of 10 mM or less.

The inventors have found that a recombinant yeast having the genes as described above is particularly sensitive towards acetic acid, as compared to non-recombinant yeasts. They have surprisingly found that the ethanol yield rapidly decreases when the composition contains more than 10 mM undissociated acetic acid, and that in order to avoid or lessen the negative effect of acetic acid the process should be performed with a composition having an amount of undissociated acetic acid of 10 mM or less, preferably 9mM or less, 8 mM or less, 7 mM or less, 6 mM or less, 5 mM or less, 4 mM or less, 3 mM or less, 2 mM or less, 1 mM or less.

In an embodiment the composition has an initial undissociated acetic acid of 10 mM or less. In another embodiment, the amount of undissociated acetic acid is 10 mM or less throughout the process.

The lower amount of undissociated acetic acid is less important. In one embodiment, the composition is free of undissociated acetic acid.

In an embodiment, the lower limit of the amount of undissociated acetic acid is 500 or more, 55 μM or more, 60 μM or more, 70 μM or more, 80 μM or more, 900 or more, 100 μM or more. The recombinant yeast used in the process of the invention comprises a gene encoding an acetylating acetaldehyde dehydrogenase, which allows the yeast to convert acetic acid, which may be present in both lignocellulosic hydrolysates and in corn starch hydrolysates, to ethanol. Although the recombinant yeast used in the process of the invention should in principle be able to consume acetic acid, the inventors have surprisingly found that there is often a residual amount of acetic acid in the fermentation media which remains unconverted. This residual amount of acetic acid may be as large as several millimolar. The inventors found that yeast requires a minimum concentration of undissociated acetic acid of at least 50 μM. Below this concentration, the consumption of acetic acid decreases, even if there is a considerable amount of dissociated acetic acid present in the fermentation media.

The skilled person appreciates that the amount of undissociated acetic acid depends inter alia on the total amount of acetic acid in the composition (protonated and dissociated) as well on the pH.

In one embodiment the amount of undissociated acetic acid is maintained at a value of at 10 mM by adjusting the pH, e.g. by adding a base.

The process may comprise the step of monitoring the pH. The pH of the composition is preferably kept between 4.2 and 5.2, preferably between 4.5 and 5.0. The lower pH is preferably such that the amount of undissociated acetic acid is 10 mM or less, which inter alia depends on the total amount of acetic acid in the composition.

The skilled person knows how to provide or select a composition having an amount of undissociated acetic acid 10 mM or less. For example, he/she may measure the amount of undissociated acetic acid in a composition and select only those compositions which have an amount of undissociated acetic acid of 10 mM or less.

Alternatively, if the amount of undissociated acetic acid in a composition exceeds 10 mM, the process may comprise, prior to the fermentation step, adding a base (such as NaOH or KOH) until the amount of undissociated acetic acid in a composition has reached a value of 10 mM or less.

The amount of undissociated acetic acid may be analysed by HPLC. HPLC generally measures all acetic acid (i.e. both undissociated, i.e. protonated form and dissociated form of acetic acid) because the mobile phase is typically acidified. In order to measure the amount of undissociated acetic acid in the composition, a suitable approach is to measure the (total) amount of acetic acid of the composition as-is, measure the pH of the composition, and calculate the amount of undissociated acetic acid using the pKa of acetic acid.

### EXAMPLES

### Material and Methods

### General Molecular Biology Techniques

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

### Media

Media which can be used in the experiments are YEPh-medium (10 g/l yeast extract, 20 g/l phytone) and solid YNB-medium (6.7 g/l yeast nitrogen base, 15 g/l agar), supplemented with sugars as indicated in the examples. For solid YEPh medium, 15 g/l agar is added to the liquid medium prior to sterilization. In the microaerobic or anaerobic cultivation experiments, Mineral Medium can be used. The composition of Mineral Medium is described by Verduyn et al., (Yeast, 1992, volume 8, pp. 501-517). Ammonium sulphate is replaced by 2.3 g/l urea as a nitrogen source. Initial pH of the medium was 4.6. In addition, for micro-/anaerobic experiments, ergosterol (0.01 g/L), Tween80 (0.42 g/L) and sugars (as indicated in examples) are added. As industrial reference medium for fermentation experiments, ‘corn mash’ can be used. This is prepared by mixing 30% w/w ground corn solids (Limagrain Westhove Maize L3) with demineralized water, adjusting the pH to 5.5 with 2M H2SO4, addition of 0.02% w/w alpha-amylase (Termamyl, Novozymes) and incubating for 4 hours at 80° C. in a rotary shaker (150 RPM). After cooling down, urea (1.00-1.25 g/L) is added as N-source and pH is adjusted to 4.5 using 2M H2SO4. 0.16 g/kg glucoamylase (Spirizyme, Novozymes) is added at the start of fermentation.

### Micro-/Anaerobic Cultivations

Strains are semi-aerobically propagated in a 100 mL Erlenmeyer shake flask without baffle and with foam plug with 10 mL Mineral Medium supplemented with 20 g/L glucose. Shake flasks are incubated 24 h at 30° C. at a shaking speed of 280 rpm. Pre-cultured cells are pelleted, washed and re-suspended with 1 culture volume sterilized water. A volume of re-suspended culture containing sufficient cell mass to inoculate the main fermentation medium to 75 mg of yeast (dry weight) per liter (see further below), is pelleted and re-suspended into main fermentation medium. Fermentation experiments are performed in an Alcoholic Fermentation Monitor (AFM, Applikon, Delft, The Netherlands), using 500 ml bottles filled to 400 ml with Mineral Medium containing ca. 60 g/L glucose. Fermentation temperature is maintained at 32° C. and vessels are stirred at 250 rpm, the pH is not controlled during fermentation. Fermentations are run for 60 hours (corn mash) or to substrate depletion (defined media). In addition to the online recording of CO₂ production by the AFM (correlating with ethanol (EtOH)), samples are taken with an interval of 4 hours during the fermentation to monitor yeast biomass, substrate utilization and product formation. For SSF samples, 1 mL/L of a 10 g/L acarbose stock solution is added to the samples to arrest glucoamylase activity. Samples for HPLC analysis are separated from yeast biomass and insoluble components (corn mash) by passing the clear supernatant after centrifugation through a 0.2 μm pore size filter.

### HPLC Analysis

HPLC analysis is typically conducted as described in “Determination of sugars, byproducts and degradation products in liquid fraction in process sample”; Laboratory Analytical Procedure (LAP, Issue date: 12/08/2006; by A. Sluiter, B. Hames, R. Ruiz, C. Scarlata, J. Sluiter, and D. Templeton; Technical Report (NREUTP-51042623); January 2008; National Renewable Energy Laboratory.

### Example 1

### Construction of Phosphoketolase Pathway-Expressing Saccharomyces cerevisiae Strains

WO2015/148272 describes a set of recombinant Saccharomyces cerevisiae strains (listed in Table 9) which reach a higher ethanol yield per gram of glucose due to lower glycerol synthesis. The recombinant strains had as common feature an integrative plasmid (pPATHt1 (TDH_A2); targeted to the delta sequences) introduced to a variety of strains derived from industrially relevant background Fermax Gold™ (Martrex Inc.). The variety of strains were different in the sense that strains had an intact glycerol synthesis pathway (FG-pPATH1, Table 9), or lacked one of the glycerol-3-phosphate dehydrogenase isoenzymes (GPD1; FGG1-pPATH1) and had reduced copies of the other (GPD2) (FGG2::pPATH1), or lacked both glycerol-3-phosphate dehydrogenase isoenzymes (GPD1, GPD2) (FGGZ-pPATH1). Deletion of one or both copies of GPD1, GPD2 and URA3 genes in industrial diploid strain Fermax GoldTM can be accomplished with methods described in e.g. WO2015/148272.

TABLE 9
listing of (recombinant) Saccharomyces cerevisiae strains

| Strain | Genotype | Reference |
|---|---|---|
| FG | Wild type Fermax Gold ™ | WO2015/148272 |
| FG-ura | GPD1/GPD1 GPD2/GPD2 Δura3/Δura3 | WO2015/148272 |
| FGG1 | Δgpd1/Δgpd1 GPD2/GPD2 Δura3/Δura3 | WO2015/148272 |
| FGG2 | Δgpd1/Δgpd1 GPD2/Δgpd2 Δura3/Δura3 | WO2015/148272 |
| FGGZ | Δgpd1/Δgpd1 Δgpd2/Δgpd2 Δura3/Δura3 | WO2015/148272 |
| FG-pPATH1 | FG-ura pPATH1(TDH_A2)/Swal | WO2015/148272 |
| FGG1-pPATH1 | FGG1 pPATH1(TDH_A2)/Swal | WO2015/148272 |
| FGG2-pPATH1 | FGG2 pPATH1(TDH_A2)/Swal | WO2015/148272 |
| FGGZ-pPATH1 | FGGz pPATH1(TDH_A2)/Swal | WO2015/148272 |
| FG-pATH1-GRU | FG-pPATH1 int1::TPI1p-DAK1-ENO1t, ENO1p-Ec_gldA- | Example |
| | CYC1t, PRE3p-Zr_T5-TEF2t | |

The plasmid pPATH1(TDH_A2) introduced into all these strains comprised overexpression cassettes enabling heterologous expression of genes involved in the phosphoketolase pathway: Bifidobacterium animalis phosphoketolase (protein sequence SEQ ID NO: 5), Lactobacillus plantarum phosphotransacetylase (protein sequence SEQ ID NO: 10) and Salmonella enterica acetaldehyde dehydrogenase (protein sequence SEQ ID NO: 26). To construct a Saccharomyces cerevisiae strain expressing a heterologous phosphoketolase pathway one could follow the methods taught in WO2015/148272 to construct and introduce pPATH1(TDH_A2). For each of the pathway elements expressed from pPATH1(TDH_A2), one could also introduce genes encoding alternative proteins proven to be expressed in Saccharomyces cerevisiae (Table 10). For the acetaldehyde dehydrogenase pathway element one could also use bifunctional acetaldehyde/alcohol dehydrogenases. Genes encoding these enzymes are preferentially codon-optimized for expression in Saccharomyces cerevisiae as also taught in WO2015/148272. These genes can replace the respective pathway element on pPATH1(TDH_A2) by using standard molecular biology cloning techniques or by synthesizing the plasmid at a DNA synthesis provider (e.g. ATUM).

TABLE 10
Alternative proteins for phosphoketolase pathway

| | | Protein | |
|---|---|---|---|
| | | sequence | Identity to |
| Pathway element | Donor organism | (SEQ ID NO) | ref seq (%) |
| phoshoketolase | Bifidobacterium animalis | 5 (ref) | 100 |
| | Bifidobacterium adolescentis | 6 | 85 |
| | Bifidobacterium lactis | 7 | 99 |
| | Leuconostoc mesenteroides | 8 | 40 |
| phosphotransacetylase | Bacillus subtilis | 9 (ref) | 100 |
| | Lactobacillus plantarum | 10 | 62 |
| | Bifidobacterium adolescentis | 11 | 29 |
| | Methosarcina thermophila | 12 | 44 |
| Acetaldehyde | Salmonella enterica (AADH) | 26 (ref) | 100 |
| dehydrogenase | Escherichia coli (eutE) | 22 | 94 |
| | Lactobacillus plantarum (acdH) | 23 | 27 |
| | Listeria innocua (acdH) | 24 | 47 |
| Bifunctional | Staphylococcus aureus (adhE) | 25 (ref) | 100 |
| acetaldehyde | Escherichia coli (adhE) | 3 | 46 |
| dehydrogenase/alcohol | | | |
| dehydrogenase | | | |

In this way, phosphoketolase pathway-expressing Saccharomyces cerevisiae strains FG-pPATH1, FGG1-pPATH1, FGG2-pPATH1, FGGZ-pPATH1 or similar strains with alternative enzymes as phosphoketolase pathway elements can be constructed.

The strains reported by WO2015/148272 displayed higher ethanol yields than wild type FG (Fermax Gold™) in anaerobic cultivation experiments in test tubes on a synthetic media supplemented with ammonium sulphate, urea and 6% glucose. The highest reported ethanol yield increases were found for the strains with deletions in the glycerol synthesis pathway (FGG1::pPATH1, FGG2::pPATH2, FGGZ-pPATH1). However, FGG1-pPATH1 suffered a hit on growth and ethanol production rate compared to FG-pPATH1 which did not deviate very much from FG (Fermax Gold™ wild type) (W02015/148272, FIG. 14A and FIG. 14B). This phenotype was visible already on laboratory defined media with 6% (=60 g/L) glucose. Under actual industrial conditions for e.g. corn ethanol process, the starch-containing biomass pretreated and hydrolyzed in a simultaneous saccharification-fermentation (SSF) set-up can contain much higher glucose levels than 60 g/L, as well as variety of other corn-matrix derived solutes and depending on plant operation and hygiene level, build-up of salts and toxic compounds from applied recycle streams (e.g. fusel alcohols, organic acids). Besides the fact that these strains display hardly any glycerol production due to the GPD deletions, these strain potentially are affected in their osmotolerance and their stress response to the external environment. Therefore, combining expression of the phosphoketolase pathway with reduction of the glycerol synthesis pathway seems to be incompatible with the more stringent conditions in the actual corn ethanol process.

### Example 2

### Construction of Saccharomyces cerevisiae Strains Expressing the Phosphoketolase Pathway Combined with the Glycerol Reuptake Pathway

To circumvent issues with osmotolerance/stress tolerance due to perturbations in the glycerol synthesis pathway, one can opt to leave the genes involved in the glycerol synthesis pathway intact (GPD1, GPD2, GPP1, GPP2) in a Saccharomyces cerevisiae strain expressing the phosphoketolase pathway. Strain FG-pPATH1 was made by that configuration. Although a higher ethanol yield was observed for FG-pPATH1 in fermentations compared to respective wild type, higher ethanol yield increases were achieved with the GPD-deletion strains indicating the maximal yield benefit was not achieved with FG-pPATH1. A higher ethanol yield per gram of released sugar is pivotal in the corn ethanol industry since small margins are to be respected. To enable a higher ethanol yield than FG-pPATH1 while keeping glycerol synthesis genes intact, in FG-pPATH1 three proteins are (over)expressed constituting a glycerol reuptake pathway: a glycerol dehydrogenase (SEQ ID NO: 15), dihydroxyacetone kinase (SEQ ID NO: 4) and a glycerol transporter (SEQ ID NO: 14). The pathway enables higher ethanol yields since the formed glycerol is re-shuttled to glycolysis by glycerol dehydrogenase and dihydroxyacetone kinase. Excreted glycerol is taken up again by the glycerol transporter facilitating more glycerol to the pathway to glycolysis.

### Expression Cassette Construction

Open reading frames (ORFs), promoter sequences and terminators can be synthesized at ATUM (Menlo Park, Calif. 94025, USA). ORFs can be synthesized as codon-optimized gene sequences for expression in Saccharomyces cerevisiae. The promoter, ORF and terminator sequences are recombined by using the Golden Gate technology, as described by Engler et al (2011, Methods Mol Biol, volume 729, pp. 167-181) and references therein. The expression cassettes are cloned into a standard sub-cloning vector. The plasmids (listed below) containing the expression cassettes encoding the components of the glycerol re-uptake pathway are:
pDB1332 (SEQ ID NO: 27) bearing expression cassette for glycerol dehydrogenase (EC 1.1.1.6) E. coli gldA under control of S. cerevisiae ENO1 promoter and S. cerevisiae CYC1 terminator;
pDB1333 (SEQ ID NO: 28) bearing expression cassette for dihydroxyacetone kinase (EC 2.7.1.29, EC 2.7.1.28) S. cerevisiae DAK1 under control of S. cerevisiae TPI1 promoter and S. cerevisiae ENO1 terminator;
pDB1336 (SEQ ID NO: 29) bearing expression cassette for glycerol transporter Z. rouxii ZYRO0E01210p (here forth referenced as Zr_T5 or T5) under control of S. cerevisiae PRE3 promoter and S. cerevisiae TEF2 terminator.

### Strain Construction

Strain construction can be done as described in WO2013/144257 and WO2016/110512. WO2013/144257 describes the techniques enabling the construction of expression cassettes from various genes of interest in such a way, that these cassettes are combined into a pathway and integrated in a specific locus of the yeast genome upon transformation of this yeast. WO2016/110512 describes the use of a CRISPR-Cas9 system for integration of expression cassettes into the genome of a host cell, in this case S. cerevisiae. Firstly, a low-copy expression vector bearing a codon-optimized gene encoding Streptococcus pyogenes Cas9 is introduced to the strain. Upon introduction of an in vivo assembled gRNA-expressing plasmid and repair DNA fragments the intended modifications are made. Firstly, an integration site in the yeast genome is selected. DNA fragments of approximately 500 bp of the up- and downstream parts of the integration locus are amplified by PCR using primers introducing connectors to the generated PCR products. These connectors (50 bp in size) allow for correct in vivo recombination of the pathway upon transformation in yeast. Secondly, the genes of interest, are amplified by PCR, incorporating a different connector (compatible with the connector on the of the neighboring biobrick) at each flank. Upon transformation of yeast cells with the DNA fragments, in vivo recombination and integration into the genome takes place at the desired location. This technique facilitates parallel testing of multiple genetic designs, as one or more genes from the pathway can be replaced with (an)other gene(s) or genetic element(s), as long as that the connectors that allow for homologous recombination remain constant and compatible with the preceeding and following biobrick in the design (WO2013/144257). As mentioned above, in a first transformation round, pCSN061 being a G418-selectable episomal plasmid bearing the S. pyogenes Cas9 expression cassette (WO2016/110512) is introduced to yeast. FG-pPATH1 is transformed with 500 ng of pCSN061. Correct transformants are selected on solid agar YNB medium supplemented with 2% w/v glucose and with 200 micrograms per milliliter G418 (Invivogen). Subsequently, several transformants can be re-streaked on YNB agar medium supplemented with 2% w/v glucose and G418 (200 micrograms per milliliter) to obtain pure colonies. Selecting one or a pool of colonies results in a FG-pPATH1 strain expressing Cas9 (FG-pPATH1-pCSN061) necessary for the next intended genetic modification.

gRNA Expression Cassette

Integration site: the expression cassettes are targeted at the INT1 locus. The INT1 integration site is a non-coding region between NTR1 (YOR071c) and GYP1 (YOR070c) located on chromosome XV of S. cerevisiae. The guide sequence to target INT1 is designed with a gRNA designer tool (https://www.dna20.com/eCommerce/cas9/input).

The gRNA expression cassette (as described by DiCarlo et al., Nucleic Acids Res. 2013; pp.1-8) can be ordered as synthetic DNA cassette (gBLOCK) at Integrated DNA Technologies (Leuven, Belgium) (INT1 gBLOCK; SEQ ID NO: 30).

gRNA-Recipient Plasmid Backbone

In vivo assembly of the gRNA expression plasmid is subsequently completed by co-transforming a linear PCR fragment derived from yeast vector pRN1120-RFP-gRNA(A). pRN1120-RFP-gRNA(A) is a multi-copy yeast shuttling vector that contains a functional natMX marker cassette conferring resistance against nourseotricin (NTC) (SEQ ID NO: 31). The backbone of this plasmid is based on pRS305 (Sikorski and Hieter, Genetics 1989, vol. 122, pp. 19-27), including a functional 2-micron ORI sequence, functional natMX marker cassette, and a RFP expression cassette to be able to track colonies that harbor the plasmid based on fluorescence or by pink to purple coloration of the colonies visible by eye.

Second Transformation Round with Specified DNA Fragments Upon Assembly Comprising Glycerol Reuptake Pathway Designs

In a second transformation round strain FG-pPATH1 expressing Cas9 (FG-pPATH1-pCSN061) is transformed with the following fragments resulting in the assembly of the glycerol reuptake pathway:
1) a PCR fragment (5′-INT1) which can be generated with primers BoZ-783 (SEQ ID NO: 32) and DBC-18463 (SEQ ID NO: 33) with genomic DNA of strain FG-pPATH1 as template;
2) a PCR fragment (DAK1) which can be generated with primers DBC-14041 (SEQ ID NO: 34) and DBC-14042 (SEQ ID NO: 35) using pDB1333 (SEQ ID NO: 28) as template;
3) a PCR fragment (gIdA) which can be generated with primers DBC-14043 (SEQ ID NO: 36) and DBC-14044 (SEQ ID NO: 37) using pDB1332 (SEQ ID NO: 27) as template;
4) a PCR fragment (T5) which can be generated with primers DBC-14046 (SEQ ID NO: 38) and DBC-14048 (SEQ ID NO: 39) using pDB1336 (SEQ ID NO: 29) as template;
5) a PCR fragment (3′-INT1) which can be generated with primers DBC-18464 (SEQ ID NO: 40) and BoZ-788 (SEQ ID NO: 41) using genomic DNA of strain FG-pPATH1 as template;
6) a PCR fragment (BB-1120RG) generated with a forward primer DBC-13664 (SEQ ID NO: 42) and a reverse primer DBC-13891 (SEQ ID NO: 43) using pRN1120-RFP-gRNA(A) (SEQ ID NO: 31) as template;
7) a PCR fragment (gRNA-INT1) which can be generated with primers DBC-13773 (SEQ ID NO: 44) and DBC-13774 (SEQ ID NO: 45) using INT1 gRNA (SEQ ID NO: 30) as template;

Transformants are selected on YNB agar medium supplemented with 2% w/v glucose and 200 micrograms G418/ml and 200 micrograms NTC/ml. Diagnostic PCR is performed to confirm the correct assembly and integration at the INT1 locus of the glycerol reuptake pathway in the strain (see Table 1 for genotype). A correct colony is selected and designated as FG-pPATH1-GRU.

### Example 3

### Fermentation Experiments on Synthetic Medium Supplemented with 60 g/L Glucose

### Propagation of Strains

Strains FG (Fermax Gold™ wild type), FG-pPATH1 and FG-pPATH1-GRU are pre-grown at 30° C. and 280 rpm overnight under semi-aerobic conditions in Mineral Medium supplemented with 20 g/L glucose.

### Preparation of Germentation Experiment

The following day, the optical density at 600 nm is determined and cells are spun down by centrifugation. Four hundred ml of Mineral Medium containing approximately 60 grams of glucose per liter is inoculated with one the abovementioned strains to 0.075 g/L (dry weight). At specific time intervals samples are taken in order to measure biomass, residual sugars, glycerol and acetic acid, as well as the formation of ethanol.

### Results Fermentation Experiment

The glycerol yield on glucose of strains FG-pPATH1 and FG-pPATH1-GRU are expected to be 30-40%, and 70-80%, respectively, lower compared to the reference strain FG (Table 11). The phosphoketolase pathway-expressing strain FG-pPATH1 is expected to produce 4% more ethanol compared to the reference strain (as also shown by WO2015/148272). Even more, the additional re-shuttling of formed glycerol through the glycerol-reuptake pathway (T5-gldA-DAK1) (Table 11) by strain FG-pPATH1-GRU is expected to result in a further increase towards ca. 6% or even higher in ethanol yield compared to the reference strain on ca. 60 g/L glucose in the experiments in this example.

TABLE 11
Fermentation characteristics of strains
FG, FG-pPATH1, FG-pPATH1-GRU on
Mineral Medium supplemented with ca. 60 g/L glucose.

| Strain | FG | FG-pPATH1 | FG-pPATH1-GRU |
|---|---|---|---|
| Relevant genotype | Wild type | PKL, PTA, | PKL, PTA, AADH, |
| | | AADH ↑ | gldA, DAK1, T5 |
| Y glycerol/glucose (g/g) | 100% | 60-70% | 20-30% |
| Y EtOH/glucose (g/g) | 100% | >100% | 102%-120% |
| Y biomass/glucose (g/g) | 100% | 90-100% | 50-70% |
| Ratio glycerol produced/ | 100% | 60-80% | 30-50% |
| biomass (mmol/gₓ) | | | |

### Example 4

### Fermentation Experiment on Corn Mash in SSF Mode Propagation of Strains

Strains FG (Fermax Gold™ wild type), FG-pPATH1, FGG1-pPATH1 and FG-pPATH1-GRU are pre-grown at 30° C. and 280 rpm overnight under semi-aerobic conditions in Mineral Medium supplemented with 20 g/L glucose.

### Preparation of Fermentation Experiment

The following day, the optical density at 600 nm is determined and cells are spun down by centrifugation. Four hundred ml of Mineral Medium containing approximately 60 grams of glucose per liter is inoculated with one the abovementioned strains to 0.075 g/L (dry weight). At specific time intervals samples are taken in order to measure free glucose, glycerol and acetic acid, as well as the formation of ethanol.

### Results Fermentation Experiment

The glycerol yield on glucose of strains FG-pPATH1 and FG-pPATH1-GRU are expected to be 30-40%, and 70-80%, respectively, lower compared to the reference strain FG (Table 12). The phosphoketolase pathway-expressing strain FG-pPATH1 is expected to produce 1.5% more ethanol compared to the reference strain (as also shown by WO2015/148272). Although the FGG1-pPATH1 strain produces less glycerol than the FG-pPATH1, it's EtOH titer is lower due to a higher residual sugar level (reduced productivity). In contrast, the additional re-shuttling of formed glycerol through the glycerol-reuptake pathway (T5-gldA-DAK1) (Table 12) by strain FG-pPATH1-GRU is expected to result in a increased EtOH titer compared to both the reference and the FG-pPATH1 strains.

TABLE 12
Fermentation yields and growth characteristics of strains FG, FG-pPATH1, FGG1-pPATH1,
FG-pPATH1-GRU on corn mash with 0.16 g/kg g/kg Spirizyme within 60 hours of fermentation.

| Strain | FG | FG-pPATH1 | FGG1-pPATH1 | FG-pPATH1-GRU |
|---|---|---|---|---|
| Relevant genotype | Wild type | PKL, PTA, AADH | PKL, PTA, AADH | PKL, PTA, AADH |
| | | | Δgpd1/Δgpd1 | gldA, DAK1, T5 |
| Glycerol titer (g/kg) | 100% | 60-70% | 20-30% | 20-30% |
| Ethanol titer (g/kg) | 100% | 100% | 80-90% | 101%-105% |
| Ethanol production | 100% | 100% | 60-70% | 90-100% |
| rate | | | | |

1. A recombinant cell, optionally a yeast cell, said recombinant cell comprising:
one or more genes coding for an enzyme having glycerol dehydrogenase activity;
one or more genes coding dihydroxyacetone kinase (E.C. 2.7.1.28 and/or E.C. 2.7.1.29);
one or more genes coding for an enzyme in an acetyl-CoA-production pathway; and
one or more genes coding for an enzyme having at least NAD⁺ dependent acetylating acetaldehyde dehydrogenase activity (EC 1.2.1.10 or EC 1.1.1.2); and optionally
one or more genes coding for a glycerol transporter.

2. The Cell according to claim 1 wherein the enzyme having glycerol dehydrogenase activity is a NAD⁺ linked glycerol dehydrogenase (EC 1.1.1.6).

3. The Cell according to claim 1 wherein the enzyme having glycerol dehydrogenase activity is a NADP⁺ linked glycerol dehydrogenase (EC 1.1.1.72).

4. The recombinant cell according to claim 1 wherein the one or more genes coding for an enzyme in an acetyl-CoA-production pathway comprises:
one or more genes coding for an enzyme having phosphoketolase (PKL) activity (EC 4.1.2.9 or EC 4.1.2.22) or an enzyme having an amino acid sequence according SEQ ID NO: 5, 6, 7, or 8, or functional homologues thereof having a sequence identity of at least 50%, and/or
one or more genes coding for an enzyme having phosphotransacetylase (PTA) activity (EC 2.3.1.8) or an enzyme having an amino acid sequence according SEQ ID NO: 9, 10, 11, or 12, or functional homologues thereof having a sequence identity of at least 50%; and/or
one or more genes coding for an enzyme having acetate kinase (ACK) activity (EC 2.7.2.12), or an enzyme having an amino acid sequence according SEQ ID NO: 1 or 2, or functional homologues thereof having a sequence identity of at least 50%.

5. The recombinant cell according to claim 1 which either lacks enzymatic activity needed for the production of acetic acid from acetaldehyde or has reduced enzymatic activity needed for production of acetic acid from acetaldehyde compared to a corresponding wild type cell thereof, optionally said cell comprises a deletion or disruption of one or more endogenous genes encoding an enzyme having NAD(P)H dependent aldehyde reductase activity (EC 1.2.1.4).

6. The recombinant cell according to claim 1 wherein the one or more genes encoding an enzyme having at least NAD⁺ dependent acetylating acetaldehyde dehydrogenase activity encodes an enzyme having an amino acid sequence according to SEQ ID NO: 3, 22, 23, 24, or 25 or a functional homologue thereof having a sequence identity of at least 50%.

7. The recombinant cell according to claim 1 wherein the enzyme having at least NAD⁺ dependent acetylating acetaldehyde dehydrogenase activity catalyses reversible conversion of acetyl-Coenzyme-A to acetaldehyde and subsequent reversible conversion of acetaldehyde to ethanol.

8. The recombinant cell according to claim 7 wherein the enzyme comprises both NAD⁺ dependent acetylating acetaldehyde dehydrogenase (EC 1.2.1.10 or EC 1.1.1.2) activity and NAD⁺ dependent alcohol dehydrogenase activity (EC 1.1.1.1).

9. The recombinant cell according to claim 1 which comprises a deletion or disruption of one or more endogenous genes encoding a glycerol exporter.

10. The Cell according to claim 1 which either lacks enzymatic activity needed for production of glycerol 3-phosphate or has reduced enzymatic activity needed for production of glycerol 3-phosphate compared to a corresponding wild type (yeast) cell thereof, optionally said cell comprises a deletion or disruption of one or more endogenous genes encoding a glycerol kinase (EC 2.7.1.30).

11. The recombinant cell according to claim 1 wherein said cell either lacks enzymatic activity needed for NADH-dependent glycerol synthesis or wherein said cell has reduced enzymatic activity needed for NADH-dependent glycerol synthesis compared to a corresponding wild type (yeast) cell thereof.

12. The Cell according to any of the preceding claim 1 which comprises a deletion or disruption of one or more endogenous genes encoding a glycerol-3-phosphate dehydrogenase optionally S. cerevisiae GPD1 and GPD2which cell is optionally free of genes encoding NADH-dependent glycerol 3-phosphate dehydrogenase.

13. The recombinant cell according to claim 1 which comprises a deletion or disruption of one or more endogenous nucleotide sequences encoding a glycerol 3-phosphate phosphohydrolase, optionally S. cerevisiae GPP1 or GPP2.

14. The recombinant cell according to claim 1 which comprises one or more genes encoding a heterologous glycerol transporter represented by SEQ ID NO: 13 or 14 or a functional homologue thereof having a sequence identity of at least 60% thereof.

15. The recombinant cell according to claim 1 which is selected from the group consisting of Saccharomycetaceae, optionally from the group consisting of Saccharomyces, optionally Saccharomyces cerevisiae; Kluyveromyces, optionally Kluyveromyces marxianus; Pichia, optionally Pichia stipitis or Pichia angusta; Zygosaccharomyces, optionally Zygosaccharomyces bailii; and Brettanomyces, optionally Brettanomyces intermedius, Issatchenkia, optionally Issatchenkia orientalis and Hansenula.

16. A product comprising a cell according to claim 1 for preparation of ethanol and/or succinic acid.

17. Process for production of a fermentation product comprising:
fermenting a composition comprising a fermentable carbohydrate, optionally selected from the group of glucose, fructose, sucrose, maltose, xylose, arabinose, galactose and mannose under anaerobic conditions in the presence of a recombinant cell according to claim 1; and
recovering the fermentation product.

18. The Process according to claim 17 wherein the fermentable carbohydrate is obtained from starch, lignocellulose, and/or pectin.

19. The Process according to claim 17, wherein the starch, lignocellulose, and/or pectin is contacted with an enzyme composition, wherein one or more sugar is produced, and wherein the produced sugar is fermented to give a fermentation product, wherein the fermentation is conducted with said recombinant cell.

20. Process according to any of claim 19, wherein the fermentation product is one or more of ethanol, butanol, lactic acid, succinic acid, a plastic, an organic acid, a solvent, an animal feed supplement, a pharmaceutical, a vitamin, an amino acid, an enzyme or a chemical feedstock.

21. Process according to claim 17 wherein said composition comprises an amount of undissociated acetic acid of 10 mM or less.

22. Process according to claim 17 wherein said composition comprises an amount of undissociated acetic acid of between 50 μM and 10 mM.


SEQUENCE LISTING (45 sequences)

SEQ ID NO: 1 - protein, 409 aa, Bifidobacterium adolescentis
  MISC_FEATURE (1)..(409): B. adolescentis acetate kinase (Bado_ACK) amino acid sequence
MAKTVLVINSGSSSIKYQLVDLETGEGIASGLVEKIGEPVDGHYKHEYNGEKHELEEPIHDHEQGLKRVLGFFDEFGPKLADAGIVAVGHRVVQGGSIFPKPALVNDKTIGQVKDLAVLAPLHNGPEAKGAEVMRSLLPDVPQIFVFDSSFFFQLPKASSTYALNKEVAQQYHIRRYGAHGTSHEFISSVVPSVIGKPAEGLKQIVLHIGNGASASAEISGKPVETSMGLTPLEGLVMGGRTGDIDPAVVFHLIRNAHMSVDELDTLFNKRSGMMGLTGFGDLREVHRLVEEGNEDAKLALDIYVHRIVGYIGNYTAQMGGVDVITFTAGVGENDDVVRKMVCDKLAPFGVKLDEEKNATRSKEPRIISTPDSAVTICVIPTNEELAIARKSAAIAEEGKDSYGNVFSK

SEQ ID NO: 2 - protein, 420 aa, Aspergillus nidulans
  MISC_FEATURE (1)..(420): A. nidulans ACK (Ani_ACK) amino acid sequence
MPRKSILSVNAGSSSVKITFYSYTKTPSVIATAQVSGITAPPATFKYSVGSKQKKEELKEKISSGPDAFKLLLHRCFTDSDLKDVASADDLAYICHRVVHGGDFESPVVINEETYHQLEDLEDLAPLHNFAALEIVRLCKKELPNVQSITFFDSSFHKSLPPYVKTYPIDQETARRNKLRKYGFHGISYSFILRSVAEYLNKPVEKTSLIALHIGSGASVCAIKDGKSIDTSMGLTPLAGLPGATRSGDIDPSLVFHYTNEAGKLSPASTKEMHISTAEEILNKKSGWKVLTGTTDFSQIAVEDPPSEQHKLAFDILVDRIVGYIGNYYVKLDGQVEGIVFAGGIGEKSALLRKAVIEQTRCLGFAVDPEKNQHGPGDDETVVDITASGRSDVKRVFICQTDEQFEMAYNCTKTQGLDKQ

SEQ ID NO: 3 - protein, 891 aa, Escherichia coli
  MISC_FEATURE (1)..(891): E. coli bifunctional NAD+ dependent acetylating acetaldehyd/alcohol dehydrogenase (E. coli adhE) amino acid sequence
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 4 - protein, 584 aa, Saccharomyces cerevisiae
  MISC_FEATURE (1)..(584): S. cerevisiae dihydroxyacetone kinase (S. cerevisiae DAK1) amino acid sequence
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 5 - protein, 825 aa, Bifidobacterium animalis
  MISC_FEATURE (1)..(825): B. animalis xylulose-5P/fructose-6P phosphoketolase (Bani_XFP.orf) amino acid sequence
MTNPVIGTPWQKLDRPVSEEAIEGMDKYWRVANYMSIGQIYLRSNPLMKEPFTRDDVKHRLVGHWGTTPGLNFLLAHINRLIADHQQNTVFIMGPGHGGPAGTAQSYIDGTYTEYYPNITKDEAGLQKFFRQFSYPGGIPSHFAPETPGSIHEGGELGYALSHAYGAIMDNPSLFVPCIIGDGEAETGPLATGWQSNKLVNPRTDGIVLPILHLNGYKIANPTILARISDEELHDFFRGMGYHPYEFVAGFDNEDHLSIHRRFAELFETIFDEICDIKAAAQTDDMTRPFYPMLIFRTPKGWTCPKFIDGKKTEGSWRAHQVPLASARDTEAHFEVLKGWMESYKPEELFNADGSIKEDVTAFMPKGELRIGANPNANGGRIREDLKLPELDQYEITGVKEYGHGWGQVEAPRSLGAYCRDIIKNNPDSFRVFGPDETASNRLNATYEVTKKQWDNGYLSALVDENMAVTGQVVEQLSEHQCEGFLEAYLLTGRHGIWSSYESFVHVIDSMLNQHAKWLEATVREIPWRKPISSVNLLVSSHVWRQDHNGFSHQDPGVTSVLLNKTFNNDHVTNIYFATDANMLLAIAEKCFKSTNKINAIFAGKQPAATWITLDEVRAELEAGAAEWKWASNAKSNDEVQVVLAAAGDVPTQEIMAASDALNKMGIKFKVVNVVDLIKLQSSKENDEAMSDEDFADLFTADKPVLFAYHSYAQDVRGLIYDRPNHDNFTVVGYKEQGSTTTPFDMVRVNDMDRYALQAKALELIDADKYADKINELNEFRKTAFQFAVDNGYDIPEFTDWVYPDVKVDETSMLSATAATAGDNE

SEQ ID NO: 6 - protein, 825 aa, Bifidobacterium adolescentis
  MISC_FEATURE (1)..(825): B. adolescentis xylulose-5P/fructose-6P phosphoketolase (Bado_XFP.orf) amino acid sequence
MTSPVIGTPWKKLNAPVSEEAIEGVDKYWRAANYLSIGQIYLRSNPLMKEPFTREDVKHRLVGHWGTTPGLNFLIGHINRLIADHQQNTVIIMGPGHGGPAGTAQSYLDGTYTEYFPNITKDEAGLQKFFRQFSYPGGIPSHYAPETPGSIHEGGELGYALSHAYGAVMNNPSLFVPAIVGDGEAETGPLATGWQSNKLINPRTDGIVLPILHLNGYKIANPTILSRISDEELHEFFHGMGYEPYEFVAGFDNEDHLSIHRRFAELFETVFDEICDIKAAAQTDDMTRPFYPMIIFRTPKGWTCPKFIDGKKTEGSWRSHQVPLASARDTEAHFEVLKNWLESYKPEELFDENGAVKPEVTAFMPTGELRIGENPNANGGRIREELKLPKLEDYEVKEVAEYGHGWGQLEATRRLGVYTRDIIKNNPDSFRIFGPDETASNRLQAAYDVTNKQWDAGYLSAQVDEHMAVTGQVTEQLSEHQMEGFLEGYLLTGRHGIWSSYESFVHVIDSMLNQHAKWLEATVREIPWRKPISSMNLLVSSHVWRQDHNGFSHQDPGVTSVLLNKCFNNDHVIGIYFPVDSNMLLAVAEKCYKSTNKINAIIAGKQPAATWLTLDEARAELEKGAAEWKWASNVKSNDEAQIVLAATGDVPTQEIMAAADKLDAMGIKFKVVNVVDLVKLQSAKENNEALSDEEFAELFTEDKPVLFAYHSYARDVRGLIYDRPNHDNFNVHGYEEQGSTTTPYDMVRVNNIDRYELQAEALRMIDADKYADKINELEAFRQEAFQFAVDNGYDHPDYTDWVYSGVNTNKQGAISATAATAGDNE

SEQ ID NO: 7 - protein, 825 aa, Bifidobacterium lactis
  MISC_FEATURE (1)..(825): B. lactis xylulose-5P/fructose-6P phosphoketolase (Blac_XFP.orf) amino acid sequence
MTNPVIGTPWQKLDRPVSEEAIEGMDKYWRVANYMSIGQIYLRSNPLMKEPFTRDDVKHRLVGHWGTTPGLNFLLAHINRLIADHQQNTVFIMGPGHGGPAGTAQSYIDGTYTEYYPNITKDEAGLQKFFRQFSYPGGIPSHFAPETPGSIHEGGELGYALSHAYGAIMDNPSLFVPCIIGDGEAETGPLATGWQSNKLVNPRTDGIVLPILHLNGYKIANPTILARISDEELHDFFRGMGYHPYEFVAGFDNEDHLSIHRRFAELFETIFDEICDIKAAAQTDDMTRPFYPMLIFRTPKGWTCPKFIDGKKTEGSWRAHQVPLASARDTEAHFEVLKGWMESYKPEELFNADGSIKEDVTAFMPKGELRIGANPNANGGRIREDLKLPELDQYEITGVKEYGHGWGQVEAPRSLGAYCRDIIKNNPDSFRVFGPDETASNRLNATYEVTKKQWDNGYLSALVDENMAVTGQVVEQLSEHQCEGFLEAYLLTGRHGIWSSYESFVHVIDSMLNQHAKWLEATVREIPWRKPISSVNLLVSSHVWRQDHNGFSHQDPGVTSVLLNKTFNNDHVTNIYFATDANMLLAIAEKCFKSTNKINAIFAGKQPAATWITLDEARAELEAGAAEWKWASNAKSNDEVQVVLAAAGDVPTQEIMAASDALNKMGIKFKVVNVVDLIKLQSSKENDEAMSDEDFADLFTADKPVLFAYHSYAQDVRGLIYDRPNHDNFTVVGYKEQGSTTTPFDMVRVNDMDRYALQAKALELIDADKYADKINELNEFRKTAFQFAVDNGYDIPEFTDWVYPDVKVDETSMLSATAATAGDNE

SEQ ID NO: 8 - protein, 813 aa, Leuconostoc mesenteroides
  MISC_FEATURE (1)..(813): L. mesenteroides xylulose-5P/fructose-6P phosphoketolase (Lmes_XFP.orf) amino acid sequence
MADFDSKEYLELVDKWWRATNYLSAGMIFLKSNPLFSVTNTPIKAEDVKVKSIGHWGTISGQTFLYAHANRLINKYGLNMFYVGGPGHGGQVMVTNAYLDGAYTEDYPEITQDIEGMSHLFKRFSFPGGIGSHMTAQTPGSLHEGGELGYSLSHAFGAVLDNPDQVAFAVVGDGEAETGPSMASWHSIKFLNAKNDGAVLPVLDLNGFKISNPTIFSRMSDEEITKFFEGLGYSPRFIENDDIHDYATYHQLAANILDQAIEDIQAIQNDARENGKYQDGEIPAWPVIIARLPKGWGGPTHDASNNPIENSFRAHQVPLPLEQHDLATLPEFEDWMNSYKPEELFNADGSLKDELKAIAPKGDKRMSANPITNGGADRSDLKLPNWREFANDINDNTRGKEFADSKRNMDMATLSNYLGAVSQLNPTRFRFFGPDETMSNRLWGLFNVTPRQWMEEIKEPQDQLLSPTGRIIDSQLSEHQAEGWLEGYTLTGRVGIFASYESFLRVVDTMVTQHFKWLRHASEQAWRNDYPSLNLIATSTAFQQDHNGYTHQDPGMLTHLAEKKSNFIREYLPADGNSLLAVQERAFSERHKVNLLIASKQPRQQWFTVEEAEVLANEGLKIIDWASTAPSGDVDITFASAGTEPTIETLAALWLINQAFPDVKFRYVNVVELLRLQKKSEPNMNDERELSAEEFNKYFQADTPVIFGFHAYENLIESFFFERKFTGDVYVHGYREDGDITTTYDMRVYSHLDRFHQAKEAAEILSANGKIDQAAADTFIAKMDDTLAKHFQVTRNEGRDIEEFTDWTWSPLK

SEQ ID NO: 9 - protein, 323 aa, Bacillus subtilis
  MISC_FEATURE (1)..(323): B. subtilis phosphotransacetylase (Bs_PTA.orf) amino acid sequence
MADLFSTVQEKVAGKDVKIVFPEGLDERILEAVSKLAGNKVLNPIVIGNENEIQAKAKELNLTLGGVKIYDPHTYEGMEDLVQAFVERRKGKATEEQARKALLDENYFGTMLVYKGLADGLVSGAAHSTADTVRPALQIIKTKEGVKKTSGVFIMARGEEQYVFADCAINIAPDSQDLAEIAIESANTAKMFDIEPRVAMLSFSTKGSAKSDETEKVADAVKIAKEKAPELTLDGEFQFDAAFVPSVAEKKAPDSEIKGDANVFVFPSLEAGNIGYKIAQRLGNFEAVGPILQGLNMPVNDLSRGCNAEDVYNLALITAAQAL

SEQ ID NO: 10 - protein, 325 aa, Lactobacillus plantarum
  MISC_FEATURE (1)..(325): L. plantarum phosphotransacetylase (Lpla_PTA.orf) amino acid sequence
MDLFESLSQKITGQDQTIVFPEGTEPRIVGAAARLAADGLVKPIVLGATDKVQAVAKDLNADLTGVQVLDPATYPAEDKQAMLDSLVERRKGKNTPEQAAKMLEDENYFGTMLVYMGKADGMVSGAVHPTGDTVRPALQIIKTKPGSHRISGAFIMQKGEERYVFADCAINIDPDADTLAEIATQSAATAKVFDIDPKVAMLSFSTKGSAKGDMVTKVQEATAKAQAAAPELAIDGEMQFDAAFVEKVGLQKAPGSKVAGHANVFVFPELQSGNIGYKIAQRFGHFEAVGPVLQGLNKPVSDLSRGCSEEDVYKVAIITAAQGLA

SEQ ID NO: 11 - protein, 556 aa, Bifidobacterium adoloscentis
  MISC_FEATURE (1)..(556): B. adoloscentis phosphotransacetylase (Bado_PTA.orf) amino acid sequence
MSFTSVTIISPEAANGRNVVALGVTKTLAAAGKTGVFRPAVCRKDTFTDVLIEASNAGLSREQSVGVCPKRARNDKEGSRADIVAAYTQAVETARPDAMVIVGTDRSAVNDPAMFSFNADVAADLQSPVLLAVCTIERTPEQVKSTVEASTKVIEDAGSKVVGVFITGCDDTQPNPLKACFVDYPVPVWTLPAVDFNDDDAISKADEAFATNVDAVELTVALESPFDAPTTPYAFQYGLLGKAKADKKTIVLPEGNEDRIIKAADYLLERDIVDLIIVGDENAILARGQELGLKSLGKAKFQAKDDETVLEPMVAKLCELRAKKGMTEEQARKQLADDSYFGTMLVVMGMADGLVSGSVNSTANTVRPALQVIKTKPGTSLVSGAFLMCFKDHAAVFADCAINLNPNAEQLAEIAIQSAETAKAFGLEPKVGMLSYSTLGSGKGPDVDLVEEATTIVKDKAPDLAVVGSIQFDAAWSPTVAATKAKGDPVAGHVNVFVFPDLCAGNIAYKAVQRSSGAAAVGPVLQGLNRPVNDLSRGATVQDIINTIALTAIEAQ

SEQ ID NO: 12 - protein, 333 aa, Methosarcina thermophila
  MISC_FEATURE (1)..(333): M. thermophila phosphotransacetylase (Mthe_PTA.orf) amino acid sequence
MVTFLEKISERAKKLNKTIALPETEDIRTLQAAAKILERGIADIVLVGNEADIKALAGDLDLSKAKIVDPKTYEKKDEYINAFYELRKHKGITLENAAEIMSDYVYFAVMMAKLGEVDGVVSGAAHSSSDTLRPAVQIVKTAKGAALASAFFIISVPDCEYGSDGTFLFADSGMVEMPSVEDVANIAVISAKTFELLVQDVPKVAMLSYSTKGSAKSKLTEATIASTKLAQELAPDIAIDGELQVDAAIVPKVAASKAPGSPVAGKANVFIFPDLNCGNIAYKIAQRLAKAEAYGPITQGLAKPINDLSRGCSDEDIVGAVAITCVQAAAQDK

SEQ ID NO: 13 - protein, 291 aa, Danio rerio
  MISC_FEATURE (1)..(291): D. rerio aquaporin 9 (Drer_T3) amino acid sequence
MEYLENIRNLRGRCVLRRDIIREFLAELLGTFVLILFGCGSVAQTVLSREAKGQLLTIHFGFTLGVMLAVYMAGGVSGGHVNPAVSLAMVVLRKLPLKKFPVYVLAQFLGAFFGSCAVYCLYYDAFTEFANGELAVTGPNVTAGIFASYPREGLSLLNGFIDQVIGAGALVLCILAVVDKKNIGAPKGMEPLLVGLSILAIGVSMALNCGYPINPARDLGPRLFTAIAGWGLTVFSAGNGWWWVPVVGPMVGGVVGAAIYFLMIEMHHPENDKNLEDDNSLKDKYELNTVN

SEQ ID NO: 14 - protein, 592 aa, Zygosaccharomyces rouxii
  MISC_FEATURE (1)..(592): Z. rouxii ZYRO0E01210p (Zrou_T5) amino acid sequence
MGKRTQGFMDYVFSRTSTAGLKGARLRYTAAAVAVIGFALFGYDQGLMSGLITGDQFNKEFPPTKSNGDNDRYASVIQGAVTACYEIGCFFGSLFVLFFGDAIGRKPLIIFGAIIVIIGTVISTAPFHHAWGLGQFVVGRVITGVGTGFNTSTIPVWQSEMTKPNIRGAMINLDGSVIAFGTMIAYWLDFGFSFINSSVQWRFPVSVQIIFALVLLFGIVRMPESPRWLMAKKRPAEARYVLACLNDLPENDDAILAEMTSLHEAVNRSSNQKSQMKSLFSMGKQQNFSRALIASSTQFFQQFTGCNAAIYYSTVLFQTTVQLDRLLAMILGGVFATVYTLSTLPSFYLVEKVGRRKMFFFGALGQGISFIITFACLVNPTKQNAKGAAVGLYLFIICFGLAILELPWIYPPEIASMRVRAATNAMSTCTNWVTNFAVVMFTPVFIQTSQWGCYLFFAVMNFIYLPVIFFFYPETAGRSLEEIDIIFAKAHVDGTLPWMVAHRLPKLSMTEVEDYSQSLGLHDDENEKEEYDEKEAEANAALFQVETSSKSPSSNRKDDDAPIEHNEVQESNDNSSNSSNVEAPIPVHHNDP

SEQ ID NO: 15 - protein, 367 aa, Escherichia coli
  MISC_FEATURE (1)..(367): E. coli glycerol dehydrogenase (Ec_gldA) amino acid sequence
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 16 - protein, 365 aa, Klebsiella pneumoniae
  MISC_FEATURE (1)..(365): K. pneumoniae glycerol dehydrogenase (Kpne_gldA) amino acid sequence
MLKVIQSPAKYLQGPDAAVLFGQYAKNLAESFFVIADDFVMKLAGEKVVNGLQSHDIRCHAERFNGECSHAEINRLMAILQKQGCRGVVGIGGGKTLDTAKAIGYYQKLPVVVIPTIASTDAPTSALSVIYTEAGEFEEYLIYPKNPDMVVMDTAIIAKAPVRLLVSGMGDALSTWFEAKACYDARATSMAGGQSTEAALSLARLCYDTLLAEGEKARLAAQAGVVTEALERIIEANTYLSGIGFESSGLAAAHAIHNGFTILEECHHLYHGEKVAFGTLAQLVLQNSPMDEIETVLGFCQRVGLPVTLAQMGVKEGIDAKIAAVAKATCAEGETIHNMPFAVTPESVHAAILTADLLGQQWLAR

SEQ ID NO: 17 - protein, 367 aa, Enterococcus aerogenes
  MISC_FEATURE (1)..(367): E. aerogenes glycerol dehydrogenase (Eaer_gldA) amino acid sequence
MDRIIQSPGKYIQGAGAIKRLGEYLKPLAERWLIIGDKFVLGFAEEQLRTSLGGAGLVAEIAPFGGECSQNEINRLRDIASSAQCHAVLGIGGGKTLDTAKALAHYMHLPVVVAPTIASTDAPCSALSVIYTDDGEFESYLMLPHNPNMVVVDTQIVAAAPARLLAAGIGDALATWFEARACSRSGATTMAGGKCTQAALALAELCYNTLVEEGEKAMLAAEQHVVTPALERVIEANTYLSGVGFESGGLAAAHAIHNGLTAIPDAHHFYHGEKVAFGTLTQLVLENAPVEEIETAAALCHSVGLPITLAQLDIKGDIPAKMRTVAEAACAEGETIHNMPGGACADQVYAALLVADQYGQRFLQEWE

SEQ ID NO: 18 - protein, 364 aa, Yersinia aldovae
  MISC_FEATURE (1)..(364): Y. aldovae glycerol dehydrogenase (Eaer_gldA) amino acid sequence
MLKVIQSPSKYIQGANALQSIGEFAKLLANNYFIIADDFVMKLTADTVGTSLQTCELKSHFSRFNGECSRQEIERLTVELKKYGCNGVIGIGGGKTLDTAKAIAHYQHIPVVVVPTIASTDAPTSALSVIYTEQGEFAEYLIYPKNPDIVLMDTTIIAKAPVRLLVAGMGDALSTYFEAQACFDAKAISMAGGASTLAAITLARLCYETLLAEGYKAKLAVEAGVVTEAVERIIEANTYLSGIGFESSGLAAAHAIHNGFTVLEECHHLYHGEKVAFGTLTQLVLQNSSMEEIETVLSFCQQLGLPITLAEMGVTQDLECKIRAVAQASCAEGETIHNMPFKVTADSVYAAIIVADRLGQAFLN

SEQ ID NO: 19 - protein, 572 aa, Klebsiella pneumoniae
  MISC_FEATURE (1)..(572): K. pneumoniae dihydroxyacetone kinase (Kpne_dhaK) amino acid sequence
MTTKQFQFDSDPLNSALAATAEASGLAYLPKSKVIYYPLTNDKVTLISGGGAGHEPAQTGFVGPGLLDAAVSGQIFASPSTKQIIAGVNAVKSQRGSIIIVMNYTGDVIHFGMAAEQLRSRYDYHAELVSIGDDISVNKKAGRRGLAGTVLVHKIAGHLARDGWDVGVLAEALRTTAANLATVAASLEHCTVPGRKFETELAADEMEIGMGIHNEPGVKTIKIGKVESLLDELVDKFEPSKQDFVPFNKGDEVVLLVNSLGGVSSLELHAIANIAQTKFEKVLGVKTVRLIVGNFMAAFNGPGFSLTLLNVTTTAKKGNFDVLGALDAPVSTAAWPSLQQKDKPANGGVQEEKETDSDKPAEPTGIKADGKLFKAMIESAVDDLKKEEPQITKYDTIAGDGDCGETLLAGGDGILDAIKNKKIDLDDAAGVADISHIVENSMGGTSGGLYSIFFSGLVVGIKETKAKELSVDVFAKACETALETLSKYTQARVGDRTLMDALVPFVETLSKTKDFAKAVEAARKGADETSKLPANFGRASYVNEEGLENIPDPGALGLAVIFEGLLKAWEKK

SEQ ID NO: 20 - protein, 580 aa, Yarrowia lipolytica
  MISC_FEATURE (1)..(580): Y. lipolytica dihydroxyacetone kinase (Ylip_DAK1) amino acid sequence
MDKHFINDPEVLVLDGLKSLADMNKTLTVHEEGKFIYFHDYNKKNVSVISGGGAGHEPTHSSFVGKGMLTAAVSGSIFASPSSKQIYTGIKQVESEAGTLVICKNYTGDILHFGMALEKQRTAGKKAELIAVADDVSVGRKKSGKVGRRGLSGTVLVHKIAGAAAARGLPLEAVTTIAKAAIDNLVSIGASLAHVHVPGHEPIAKEDEMKHDEMELGMGIHNEPGCKRISPIPSIDDLIAQMLKQMLDQSDKDRAYVKIEGDDEVVLLMNNLGGLSMLEFSAISHKVKEALAKEYKINPVRIFAGPFTTSLNGLGFGITLLRTTDRVKVEGEEYSLVDLIDQPVEAIGWPLCQPSDLKSKNKIGNVSIEEGQKDVKSPVTVDKEKVRQAIVNSMENLIKAEPKITKFDTMAGDGDCGTTLKRGAEGVLKFVKSDKFSDDPIRIVRDIADVIEDNMDGTSGALYAIFFHGFAKGMKDTLEKSKDISSKTWAAGLKVALDTLFKYTPARPGDSTMCDALVPFVETFVKTNDLNAAVEEARKGADATADMQAKLGRAVYVGDDVKVPDAGALGVVAIVEGFTK

SEQ ID NO: 21 - protein, 580 aa, Schizosaccharomyces pombe; identical to SEQ ID NO: 20
  MISC_FEATURE (1)..(580): S. pombe dihydroxyacetone kinase (Spom_DAK1) amino acid sequence

SEQ ID NO: 22 - protein, 467 aa, Escherichia coli
  MISC_FEATURE (1)..(467): E. coli ethanolamine utilizing protein (Ec_eutE) amino acid sequence
MNQQDIEQVVKAVLLKMQSSDTPSAAVHEMGVFASLDDAVAAAKVAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAGKIAAAIGLKVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 23 - protein, 455 aa, Lactobacillus plantarum
  MISC_FEATURE (1)..(455): L. plantarum acetaldehyde dehydrogenase (Lpla_acdH) amino acid sequence
MLKEMEETTVSRSIDRLVLNASLAANRLEVMDQSQVDQAVAAMARAAHAARGMLAAMAVEETGRGNYRDKVAKNDFAAKNVYNYIKDDKTVGIINDDPVSGVMKVAEPVGIIAGVTPVTNPTSTVIFNAMLALKTRNPIIFGFHPFAQKSCVETGRIIRDAAIASGAPKDWIQWIKTPSLEATNTLMNHPGVATIIATGGAGMVKTAYSTGKPALGVGPGNVPCFIEQTADIQQAVSDVVTSKSFDNGMICASESNLIVADQIYDQVKRELSHNGVYFVGTENFKALEATVMNLDKQAVDPKVAGQTPWQIAQWAGFDVPSDTKVLAVELPSIGGDQVLSREKLSPVLAVVHAKDTEAGFNLMKRSLALGGLGHTAALHTTDEAVMNKFALEMTACRALINVPSSQGAIGYKYDNVAPSLTLGCGTWGHNSISHNLEDWDLLNIKTVAKRLTKIR

SEQ ID NO: 24 - protein, 469 aa, Listeria innocua
  MISC_FEATURE (1)..(469): L. innocua acetaldehyde dehydrogenase (Linn_acdH) amino acid sequence
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRDSCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 25 - protein, 869 aa, Staphylococcus aureus
  MISC_FEATURE (1)..(869): S. aureus acetaldehyde/alcohol dehydrogenase (Saur_adhE) amino acid sequence
MLTIPEKENRGSKEQEVAIMIDALADKGKKALEALSKKSQEEIDHIVHQMSLAAVDQHMVLAKLAHEETGRGIYEDKAIKNLYASEYIWNSIKDNKTVGIIGEDKEKGLTYVAEPIGVICGVTPTTNPTSTTIFKAMIAIKTGNPIIFAFHPSAQESSKRAAEVVLEAAMKAGAPKDIIQWIEVPSIEATKQLMNHKGIALVLATGGSGMVKSAYSTGKPALGVGPGNVPSYIEKTAHIKRAVNDIIGSKTFDNGMICASEQVVVIDKEIYKDVTNEFKAHQAYFVKKDELQRLENAIMNEQKTGIKPDIVGKSAVEIAELAGIPVPENTKLIIAEISGVGSDYPLSREKLSPVLALVKAQSTKQAFQICEDTLHFGGLGHTAVIHTEDETLQKDFGLRMKACRVLVNTPSAVGGIGDMYNELIPSLTLGCGSYGRNSISHNVSATDLLNIKTIAKRRNNTQIFKVPAQIYFEENAIMSLTTMDKIEKVMIVCDPGMVEFGYTKTVENVLRQRTEQPQIKIFSEVEPNPSTNTVYKGLEMMVDFQPDTIIALGGGSAMDAAKAMWMFFEHPETSFFGAKQKFLDIGKRTYKIGMPENATFICIPTTSGTGSEVTPFAVITDSETNVKYPLADFALTPDVAIIDPQFVMSVPKSVTADTGMDVLTHAMESYVSVMASDYTRGLSLQAIKLTFEYLKSSVEKGDKVSREKMHNASTLAGMAFANAFLGIAHSIAHKIGGEYGIPHGRANAILLPHIIRYNAKDPQKHALFPKYEFFRADTDYADIAKFLGLKGNTTEALVESLAKAVYELGQSVGIEMNLKSQGVSEEELNESIDRMAELAFEDQCTTANPKEALISEIKDIIQTSYDYKQ

SEQ ID NO: 26 - protein, 467 aa, Salmonella enterica
  MISC_FEATURE (1)..(467): S. enterica acetaldehyde dehydrogenase (Sent_acdH) amino acid sequence
MNQQDIEQVVKAVLLKMKDSSQPASTVHEMGVFASLDDAVAAAKRAQQGLKSVAMRQLAIHAIREAGEKHARELAELAVSETGMGRVDDKFAKNVAQARGTPGVECLSPQVLTGDNGLTLIENAPWGVVASVTPSTNPAATVINNAISLIAAGNSVVFAPHPAAKKVSQRAITLLNQAVVAAGGPENLLVTVANPDIETAQRLFKYPGIGLLVVTGGEAVVDAARKHTNKRLIAAGAGNPPVVVDETADLPRAAQSIVKGASFDNNIICADEKVLIVVDSVADELMRLMEGQHAVKLTAAQAEQLQPVLLKNIDERGKGTVSRDWVGRDAGKIAAAIGLNVPDQTRLLFVETPANHPFAVTEMMMPVLPVVRVANVEEAIALAVQLEGGCHHTAAMHSRNIDNMNQMANAIDTSIFVKNGPCIAGLGLGGEGWTTMTITTPTGEGVTSARTFVRLRRCVLVDAFRIV

SEQ ID NO: 27 - DNA, 2013 bp, Artificial Sequence
  pDB1332
gtgcccgcggaaccgccagatattcattacttgacgcaaaagcgtttgaaataatgacgaaaaagaaggaagaaaaaaaaagaaaaataccgcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtgtcaaggtcaaaactgtatggaaaccttgtcacctcacttaattctagctagcctaccctgcaagtcaagaggtgtccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaaatggacagaatcatccaatctccaggtaagtacatccaaggtgctgatgttatcaacagattaggtgaatacttgaagccattggctgaaagatggttagtcgtcggtgacaaattcgttttgggtttcgctcaatccaccgtcgaaaagtctttcaaggatgctggtttggttgttgaaatcgctccattcggtggtgaatgttctcaaaatgaaattgaccgtttgagaggtattgctgaaactgctcaatgtggtgccatcttgggtattggtggtggtaagactttggacactgccaaggctttggcccacttcatgggtgttccagttgccattgctccaaccattgcttctaccgatgctccatgttctgctttgtccgttatctacaccgacgaaggtgaatttgaccgttacttgttgttgccaaacaacccaaacatggtcattgtcgacaccaagatcgttgccggtgctccagccagattattggctgccggtatcggtgatgctttggctacctggttcgaagccagagcttgttccagatctggtgctactaccatggccggtggtaaatgtactcaagctgctttagctttggctgaattgtgttacaacactttgttggaagaaggcgaaaaggctatgttggctgctgaacaacacgttgttactccagctttggaaagagtcattgaagccaacacctacttgtccggtgttggattcgaatctggtggtttagctgccgctcatgccgtccacaacggtttgactgccatcccagatgctcaccactactaccacggtgaaaaggttgctttcggtactttgactcaattagtcttggaaaacgctccagtcgaagaaatcgaaaccgttgctgctctatcccacgctgtcggtttgcctatcactttggctcaattggacatcaaggaagatgtcccagctaagatgagaattgttgctgaagctgcttgtgctgaaggtgaaaccattcacaacatgccaggtggtgccaccccagaccaagtctacgctgctttgttggttgctgaccaatacggtcaaagattcttgcaagaatgggagtaaaacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagcttcgcagtttacactctcatcgtcctc

SEQ ID NO: 28 - DNA, 2664 bp, Artificial Sequence
  pDB1333
gtgcgacacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatgtccgctaaatctttcgaagttaccgacccagtcaactcttctttgaagggttttgctttggccaacccatccattactttggtcccagaagaaaagatcttattcagaaagactgactctgacaaaattgctttgatctccggtggtggttccggtcacgaaccaacccacgctggtttcatcggtaagggtatgttgtccggtgctgtcgttggtgaaatctttgcttctccatccaccaagcaaatcttgaatgctatcagattagtcaacgaaaacgcttctggtgtcttgttgattgtcaagaactacactggtgacgtcttgcatttcggtttatctgctgaaagagctagagctttgggtattaactgtagagttgccgtcatcggtgacgatgttgctgtcggtcgtgaaaagggtggtatggttggtagacgtgctttggctggtactgtcttggttcacaagattgttggtgctttcgctgaagaatactcctccaagtacggtttagatggtactgctaaggttgccaagatcatcaacgacaacttggttaccatcggttcttctttggaccactgtaaggttccaggtagaaagttcgaatctgaattgaacgaaaagcaaatggaattgggtatgggtatccacaacgaaccaggtgttaaggtcttggacccaattccatccactgaagatttgatttccaaatacatgttgccaaagttgctagacccaaacgacaaggacagagctttcgttaagttcgatgaagatgacgaagttgttttgttggtcaacaacttgggtggtgtttctaacttcgtcatctcttctattacctccaagaccaccgatttcttaaaggaaaactacaacatcactccagtccaaaccattgccggtactttgatgacctctttcaacggtaacggtttctccatcaccttgttgaatgccaccaaagctaccaaggctttgcaatctgatttcgaagaaatcaaatccgtcttagatttgttgaacgccttcaccaacgccccaggttggccaattgctgacttcgaaaagacctctgctccatctgttaacgatgacttgttgcacaacgaagttactgccaaggccgtcggtacttacgatttcgacaaattcgctgaatggatgaagtctggtgctgaacaagtcatcaaatctgaaccacacatcactgaattggacaaccaagttggtgatggtgactgtggttacactttggttgctggtgtcaagggtatcactgaaaacttggacaaattgtccaaggactctttgtctcaagctgttgctcaaatttctgatttcattgaaggttccatgggtggtacttctggtggtttgtactccatcttgttgtctggtttctcccacggtttgatccaagtttgtaagtccaaggatgaacctgtcaccaaggaaattgttgccaagtctctaggtattgctttggacactttatacaagtacaccaaggccagaaagggttcttccaccatgatcgatgctttggaaccatttgtcaaggaattcactgcttctaaggacttcaacaaggctgttaaggctgctgaagaaggtgccaagtccactgctactttcgaagctaagttcggtagagcttcttacgttggtgactcttctcaagttgaagatccaggtgctgttggtttatgtgaattcttgaagggtgtccaatctgcgctttaaaagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcccctc

SEQ ID NO: 29 - DNA, 2684 bp, Artificial Sequence
  pDB1336
gtgccaaacattaatttgttctgcatactttgaacctttcagaaaataaaaaacattacgcgcatacttaccctgctcgcgaagaagagtaacactaacgcattctatgggcaattgaagacagtattcagtacaagacatagtccgtttccttgagtcaattcctatagcattatgaactagccgcctttaagagtgccaagctgttcaacaccgatcatttttgatgatttggcgtttttgttatattgatagatttcttttgaattttgtcattttcacttttccactcgcaacggaatccggtggcaaaaaagggaaaagcattgaaatgcaatctttaacagtattttaaacaagttgcgacacggtgtacaattacgataagaattgctacttcaaagtacacacagaaagttaacatgaatggaattcaagtggacatcaatcgtttgaaaaagggcgaagtcagtttaggtacctcaatgtatgtatataagaatttttcctcccactttattgtttctaaaagttcaatgaagtaaagtctcaattggccttattactaactaataggtatcttataatcacctaataaaatagaatgggtaagagaactcaaggtttcatggactacgttttctccagaacttctactgctggtttgaaaggtgccagattgagatacactgctgctgccgttgctgttatcggtttcgctttattcggttacgaccaaggtttgatgtctggtttgattaccggtgaccaattcaacaaggaattccctccaaccaaatctaacggtgacaacgacagatacgcctccgtcatccaaggtgctgtcactgcttgttacgaaattggttgtttcttcggttctttgttcgtcttattctttggtgatgccatcggtagaaagccattaatcattttcggtgccatcatcgtcatcattggtactgttatctccactgctccattccaccacgcttggggtttgggtcaatttgttgtcggtagagttatcactggtgtcggtactggtttcaacacttccaccatcccagtctggcaatctgaaatgaccaagccaaacatcagaggtgctatgattaacttggatggttccgtcattgctttcggtactatgattgcttactggttggatttcggtttctctttcatcaactcttctgttcaatggagattcccagtttccgttcaaatcatctttgctttggtcttattgttcggtattgtcagaatgccagaatctccaagatggttgatggccaagaagcgtccagctgaagccagatacgttttggcttgtttgaatgacttgccagaaaacgacgatgccatcttggccgaaatgacttctttgcacgaagctgtcaacagatcttctaaccaaaagtctcaaatgaaatctttgttctccatgggtaagcaacaaaacttctccagagctttgattgcttcctctacccaattcttccaacaattcaccggttgtaatgctgccatctactactccactgttttgttccaaaccaccgttcaattagatagattgttggctatgatcttgggtggtgtctttgctaccgtttacaccttgtccactttgccatctttctacttggttgaaaaggttggtcgtcgtaagatgttcttcttcggtgctttaggtcaaggtatttctttcattattacctttgcttgtttggtcaacccaaccaagcaaaacgctaagggtgctgctgtcggtttatacttgttcatcatctgtttcggtttggctatcttggaattgccatggatctacccaccagaaattgcttccatgagagtcagagctgctaccaacgctatgtccacctgtaccaactgggttaccaactttgccgttgtcatgttcaccccagtctttatccaaacttctcaatggggttgttacttgttcttcgctgtcatgaacttcatctacttgcctgttatcttcttcttctacccagaaactgccggtcgttctttggaagaaatcgatattattttcgctaaggctcatgttgacggtactctaccatggatggttgctcacagattaccaaagttgtccatgactgaagtcgaagattactctcaatctttgggtttgcacgacgatgaaaacgaaaaggaagaatacgacgaaaaagaagctgaagctaacgctgccttgttccaagttgaaacctcttccaagtctccatcctccaacagaaaggatgacgacgctccaattgaacacaacgaagttcaagaatctaacgacaactcctctaactcttctaacgttgaagctccaattccagttcaccacaacgacccataaagagtaataattattgcttccatataatatttttatatacctcttatttttatgtattagttaattaagtatttttatctatctgcttatcattttcttttcatatagggggggttggtgttttcttgcccatcagattgatgtcctccaactcggcactattttacaaagggtttttttgtaagagaaggagaagacagatactaaaccatacgttactcgaaacaaaaaaaaaaaaaatggaaaaagctgctatcaacaaaagacggcctcatcaaacctaaagaaaccatgtcagcgt

SEQ ID NO: 30 - DNA, 486 bp, Artificial Sequence
  INT1 gRNA gBLOCK
ctgcagcccgggggatccactagttctagagcggccgccaccgcccgcggtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctccgcggcaattaaccctcactaaagggaacaaaagctggagctccacc

SEQ ID NO: 31 - DNA, 7794 bp, Artificial Sequence
  pRN1120-RFP-gRNA(A)
tcatgtttgacagcttatcatcgataatccggagctagcatgcggccgcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttgttccagacacgacgtcagttttagagctagaaatagcaagttaaaataaggctagtccgttatcaacttgaaaaagtggcaccgagtcggtggtgctttttttgttttttatgtctcccgatatcaagcttatcgacgctttccggcatcttccagaccacagtatatccatccgcctcctgttgaggaccggtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaagaattcaaaatggtttcaaaaggtgaagaagataatatggctattattaaagaatttatgagatttaaagttcatatggaaggttcagttaatggtcatgaatttgaaattgaaggtgaaggtgaaggtagaccatatgaaggtactcaaactgctaaattgaaagttactaaaggtggtccattaccatttgcttgggatattttgtcaccacaatttatgtatggttcaaaagcttatgttaaacatccagctgatattccagattatttaaaattgtcatttccagaaggttttaaatgggaaagagttatgaattttgaagatggtggtgttgttactgttactcaagattcatcattacaagatggtgaatttatttataaagttaaattgagaggtactaattttccatcagatggtccagttatgcaaaaaaaaactatgggttgggaagcttcatcagaaagaatgtatccagaagatggtgctttaaaaggtgaaattaaacaaagattgaaattaaaagatggtggtcattatgatgctgaagttaaaactacttataaagctaaaaaaccagttcaattaccaggtgcttataatgttaatattaaattggatattacttcacataatgaagattatactattgttgaacaatatgaaagagctgaaggtagacattcaactggtggtatggatgaattatataaataatctagacaaatcgctcttaaatatatacctaaagaacattaaagctatattataagcaaagatacgtaaattttgcttatattattatacacatatcatatttctatatttttaagatttggttatataatgtacgtaatgcaaaggaaataaattttatacattattgaacagcgtccaagtaactacattatgtgcactaatagtttagcgtcgtgaagactttattgtgtcgcgaaaagtaaaaattttaaaaattagagcaccttgaacttgcgaaaaaggttctcatcaactgtttaaaagatctgagctcgcagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattc

SEQ ID NO: 32 - DNA, 19 bp, Artificial Sequence
  BoZ-783
cggcattattgtgtatggc

SEQ ID NO: 33 - DNA, 72 bp, Artificial Sequence
  DBC-18463
aatcgcaactcggatttgggaggcaaggtcggaacgcgaactttggctttcatgttatgctcctcaatcacg

SEQ ID NO: 34 - DNA, 70 bp, Artificial Sequence
  DBC-14041
aaagccaaagttcgcgttccgaccttgcctcccaaatccgagttgcgattgtgcgacacctaactacata

SEQ ID NO: 35 - DNA, 70 bp, Artificial Sequence
  DBC-14042
actcgaaagcagggaatcgaactggagttggactttcttcgggatcgggtgaggggaggaaatgagaaat

SEQ ID NO: 36 - DNA, 70 bp, Artificial Sequence
  DBC-14043
acccgatcccgaagaaagtccaactccagttcgattccctgctttcgagtgtgcccgcggaaccgccaga

SEQ ID NO: 37 - DNA, 70 bp, Artificial Sequence
  DBC-14044
tcgcttgcgaaattcggaactggcaagaatctgggagacaaacggatactgaggacgatgagagtgtaaa

SEQ ID NO: 38 - DNA, 71 bp, Artificial Sequence
  DBC-14046
agtatccgtttgtctcccagattcttgccagttccgaatttcgcaagcgagtgccaaacattaatttgttc

SEQ ID NO: 39 - DNA, 71 bp, Artificial Sequence
  DBC-14048
gaaagaaattgcgagcggatacggtggaggaaagttctgccttgtattggacgctgacatggtttctttag

SEQ ID NO: 40 - DNA, 74 bp, Artificial Sequence
  DBC-18464
ccaatacaaggcagaactttcctccaccgtatccgctcgcaatttctttcgaatgacctgttcccgacactatg

SEQ ID NO: 41 - DNA, 24 bp, Artificial Sequence
  BoZ-788
cacaagcttattcttccaaaaatc

SEQ ID NO: 42 - DNA, 70 bp, Artificial Sequence
  DBC-13664
acgctttccggcatcttccagaccacagtatatccatccgcctcctgttgaggaccggtttatcattatc

SEQ ID NO: 43 - DNA, 24 bp, Artificial Sequence
  DBC-13891
agcggccgcatgctagctccggat

SEQ ID NO: 44 - DNA, 73 bp, Artificial Sequence
  DBC-13773
cttatcgataccgtcgacctcgagggggggcccggtacccagcttttgttccgcggtctttgaaaagataatg

SEQ ID NO: 45 - DNA, 69 bp, Artificial Sequence
  DBC-13774
aaaatacaacaaataaaaaacactcaatgacctgaccatttgatggagttccgcggagacataaaaaac